(19) United States
(12) Patent Application Publication
(10) Pub. No.: US 2022/0118379 A1
(43) Pub. Date: Apr. 21, 2022

(54) Separation of Radiometals

(71) Applicants: Technical University of Denmark, Kongens Lyngby (DK); Massachusetts Institute of Technology, Cambridge, MA (US)

(72) Inventors: Fedor ZHURAVLEV, Roskilde (DK); Kristina Søborg PEDERSEN, Roskilde (DK); Jesper FONSLET, Birkerød (DK); Joseph Michael IMBROGNO, North Stonington, CT (US); Andrea ADAMO, Cambridge, MA (US); Klavs F. JENSEN, Lexington, MA (US)

(21) Appl. No.: 17/266,383

(22) PCT Filed: Aug. 6, 2019
(86) PCT No.: PCT/EP2019/071156

§ 371 Date: Feb. 5, 2021

Related U.S. Application Data
(60) Provisional application No. 62/715,471, filed on Aug. 7, 2018.
(60) Provisional application No. 62/772,803, filed on Nov. 29, 2018.

(51) Int. Cl. B01D 11/04; C01G 9/00; C01F 17/17; C01G 53/00; A61K 51/04; C22B 3/26; C22B 15/00; C22B 34/14; C22B 34/12; C22B 58/00; B01D 61/14; B01D 61/16; B01D 61/18; G21G 1/00

(52) U.S. Cl. CPC B01D 11/0415; B01D 2311/2626; C01F 17/17; C01G 53/003; A61K 51/0478; C22B 3/302; C22B 15/0084; C22B 34/14; C22B 34/1259; C22B 58/00; B01D 61/147; B01D 61/16; B01D 61/18; B01D 11/0492; G21G 1/00; B01D 2311/04; C01G 9/003

(57) ABSTRACT

Method of separation of a radiometal ion from a target metal ion, comprising a first liquid-liquid extraction step in which an organic phase comprising an extractant and an interfacial tension modifier is mixed with an aqueous phase comprising the radiometal ion and the target metal ion in order that the radiometal ion is at least partially transferred to the organic phase, followed by a first phase separation step, wherein the phase separation is carried out in flow comprising the use of a microfiltration membrane to separate the phases based on the interfacial tension between the phases such that a permeate phase passes through the membrane and a retentate phase does not.

### FIELD OF THE INVENTION

The present invention is concerned with the separation of metal ions, in particular, radiometal ions, from other metal ions in aqueous solution. In particular, the present invention relates to methods of continuous separation of radiometal ions from target metal ions from which the radiometal ions have been generated, especially to such methods that may be used in the generation of radiometals and radiopharmaceuticals for medical and veterinary use, such as in positron emission tomography (PET). In addition, the present invention relates to continuous methods of production of radiometals, optionally including recycling of the separated target metal, and methods of production of radiolabeled compounds. Apparatus for carrying out such a separation is also provided, along with the use of such apparatus in separation of metal ions.

### BACKGROUND OF THE INVENTION

Over the past several decades, positron emission tomography (PET) has become one of the best available diagnostic options for cancer¹. PET radiopharmaceuticals based on radiometals are gaining in popularity due to their ability to probe biological processes occurring on timescales from hours to days².

Radiometals such as ⁶⁸Ga, ⁸⁹Zr, ⁶⁴Cu, and ⁴⁵Ti are finding increased use in peptide and antibody-based PET radiopharmaceuticals due to their widely ranging half-lives, which allow for matching with the circulation time of the biological vector of interest, high radiolabelling yields, little or no post-labelling purification requirement, and the possibility of carrying out late-stage radiolabelling^{2,3,4}.

⁶⁸Ga is experiencing a particularly high adoption rate in clinics⁵. For example, ⁶⁸Ga-PSMA (prostate-specific membrane antigen) is emerging as the gold standard for prostate cancer diagnostics⁶, and other tracers are in development⁷. The synthesis of Ga-PSMA has been described⁸.

The convenient chelation chemistry and ready availability via the ⁶⁸Ge generator contribute to this popularity⁹. However, this has also caused the so-called “Ga rush”: all medical ⁶⁸Ga is supplied by gallium generators, which are based on ⁶⁸Ge isotopes produced by large particle accelerators^{10,11}. These generators can suffer from high prices, quality inconsistencies¹², limited shelf life and inherently low yield⁹ of ⁶⁸GaCl₃². At medical conferences, the inventors have heard clinicians comment that they are not able to source enough ⁶⁸Ga from the generators to support larger clinical trials. Accordingly, alternative sources of ⁶⁸Ga are needed.

An alternative means of production of ⁶⁸Ga is the irradiation of the stable isotope ⁶⁸Zn using a cyclotron¹³. As many hospitals have their own cyclotrons, this is potentially a convenient means of production of ⁶⁸Ga for radiotracers and the like. However, the cyclotron production of ⁶⁸Ga from ⁶⁸Zn and its separation requires a series of manual operations, entailing significant radiation exposure to the personnel carrying out those operations, and the process is not easily amenable to automation.

The production of ⁶⁸Ga from a zinc salt solution target has recently been described, in which zinc chloride¹⁴ or zinc nitrate¹⁵ is used as the solution target in a cyclotron. However, the irradiated solution of ⁶⁸Zn and ⁶⁸Ga resulting from this step still requires a semi-manual separation on two solid-phase cartridges¹⁶. Although the procedure is capable of recovering the expensive ⁶⁸Zn target material for re-use it is laborious and slow. Further, the eluted ⁶⁸Ga needs to be re-formulated before it can be used in radiolabelling. Recently⁷³, a cassette style apparatus for conducting ion exchange chromatographic separation of ⁶⁸Zn and ⁶⁸Ga has been described, in which ⁶⁸Zn can be recovered in an acetone solution, and, it is said, can be re-used. However, it is necessary for ⁶⁸Zn target solutions to be rigorously organic-free in order that they can be used as solution targets. Accordingly, there are improvements to be made in the production of ⁶⁸Ga for medical and radiolabelling purposes.

⁴⁵Ti shows promise as a PET radiometal due to its 85.7% positron branch, negligible secondary radiation, and facile production. Its three-hour half-life compares favourably with that of ⁶⁸Ga (68 min) and can allow for longer transport distances. Furthermore, the sharper PET images of ⁴⁵Ti due to its lower p endpoint energy (1.04 MeV for ⁴⁵Ti versus 1.90 MeV for ⁶⁸Ga) can be especially advantageous for small-animal PET. A number of small molecule ⁴⁵Ti compounds have been synthesised and used for PET imaging and radiotracing.

The bombardment of naturally monoisotopic scandium with low energy protons from a medical cyclotron via the ^{nat}Sc(p,n)⁴⁵Ti nuclear reaction is an attractive ⁴⁵Ti production route¹⁷⁻¹⁹. Recovery of the radiometal is the first post-production step, which, for a highly hydrolysable metal such as Ti, becomes critical. Currently, the solid phase extraction from acidic solutions on to a cation or anion exchange resin is the predominant way to separate the ⁴⁵Ti from its Sc matrix. The present inventors and others have previously used the PEG-functionalised diol²⁰, cation exchange²¹⁻²³ and hydroxamate²⁴ resins. However, as for the separation of ⁶⁸Ga, this method is capable of improvement for safety and efficiency, especially regarding re-use of the Sc target material.

Even longer transportation and post-injection imaging times are possible with ⁸⁹Zr. The ⁸⁹Zr radioisotope decays with a half-life of 3.27 days via electron capture (77%), and positron emission (23%) to ⁸⁹Y.²⁵ Since residence time of monoclonal antibodies (mAbs) in humans ranges from a few days to weeks, ⁸⁹Zr appears to be an ideal radionuclide for use in immuno-PET. Conjugated via desferrioxamine (DFO)-derived bifunctional chelators, ⁸⁹Zr-labelled Cetuximab, Trastuzumab, and J591 have been prepared and investigated pre-clinically and clinically.²⁶ The synthesis of Zr-trastuzumab has been described²⁷.

The proton bombardment of a thick target prepared from naturally monoisotopic ⁸⁹Y (^{nat}Y) at optimum energy of 14 MeV yields up to 58 MBq/μAh of the ⁸⁹Zr radionuclide. The separation of zirconium from bulk yttrium typically involves the adsorption of the radionuclide onto a hydroxamate resin followed by elution with oxalic acid.²⁸

⁸⁹Zr obtained from a solution target of Y(NO₃)₃ has been described, along with column based separation methods to isolate the ⁸⁹Zr as the hydrogen phosphate, instead of the conventional oxalate, avoidance of which is beneficial for reasons of its toxicity¹⁶.

⁶⁴Cu is the most commonly used Cu radioisotope. It has a half life of 12.7 h, and so is well suited to PET studies conducted over a 48 h period. This half life also allows for transport distances longer than for ⁶⁸Ga. ⁶⁴Cu decays 17.4% by positron emission, and has a β+ maximum energy of 0.66 MeV with average energy of 0.28 MeV, allowing for very high quality PET images. In addition, ⁶⁴Cu also decays by electron capture (43%) and by β− (43%), and so can be used as both a therapeutic and a diagnostic radionuclide. Radiopharmaceuticals based on Cu-thiosemicarbazones have been developed to measure blood flow, for example Cu-pyruvaldehyde-bis(N⁴-methylthiosemicarbazone) (Cu-PTSM), and, more recently, in the imaging of hypoxic tissues, for example Cu-diacetyl-bis(N⁴-methylthiosemicarbazone) (Cu-ASTM).⁷⁵

Extraction of metal ions from an aqueous solution and separation of one metal ion from another both present in aqueous solution has been extensively studied. There are many known methods of carrying out liquid-liquid extraction to carry out such separations. For example:
Liquid-liquid based batch separation of ⁶⁸Ga ions from ⁶⁸Zn ions comprised in an aqueous solution of protic acid using isopropyl ether as extractant, followed by back extraction with HCl; it is said that this provides high purity Ga separation from Zn compared with cation exchange²⁹.
Liquid-liquid based batch separation of ⁶⁸Ga ions from ⁶⁸Zn ions comprised in an aqueous solution of ammonia using HDEHP in cyclohexane as an extractant³⁰.
Liquid-liquid extraction based batch separation of non-radioactive gallium ion from zinc ion comprised in acidic aqueous solution. The extractants used are acidic organophosphates having bulky alkyl groups in toluene³¹.
Batch separation of gallium ion from a tertiary Ga/Bi/Zn system is described: the aqueous solution containing bismuth and gallium and zinc ions is adjusted to pH 4.5 and 0.007M sodium succinate, followed by extraction with 0.73 M 2-octylaminopyridine in chloroform for 5 minutes. This leaves the zinc(II) ions in the aqueous phase and the bismuth and gallium ions in the organic phase. The bismuth is then removed from that with 0.5 M nitric acid, leaving the gallium ion in the organic phase. Back extraction with an aqueous solution of 0.1 M EDTA then brings the gallium ion into an aqueous phase once again³².
Liquid-liquid extraction based batch separation of ⁸⁸Zr and ⁸⁹Zr from yttrium ions comprised in an aqueous solution of a protic acid, in which TPPO dissolved in chloroform, or HDEHP in chloroform are used as extractant, followed by back extraction using an oxalate, is described, with the use of TPPO in chloroform being preferred for separation of zirconium ions from yttrium ions.³³
Liquid-liquid extraction based batch separation of ⁸⁹Zr from yttrium ions comprised in an aqueous solution of a protic acid, in which di-n-butyl phosphate (DBP) dissolved in di-n-butyl ether is used as extractant, followed by back extraction with 4 M HF and a final purification on a Dowex 1×8 resin, is described.³⁴
Liquid-liquid extraction based batch separation of zirconium from yttrium ions comprised in an aqueous solution of a protic acid, in which trioctylphosphine oxide dissolved in kerosene, is described.³⁵
A study³⁶ into the selectivity of a cation exchange resin for Cu radioisotopes (in particular ⁶¹Cu) and Ni ions is described, with particular relevance to the HNO₃ concentration during the separation, which is said to be more effective and simpler than anion exchange separation of the same ions. A solvent extraction method of separation⁷⁴ is mentioned in the introduction as being very complex and leading to loss of radioactive copper. It is to be noted that the best performing solvent in this batch extraction procedure is carbon tetrachloride, whose use is not acceptable for environmental and toxicity reasons.
Batch separation of Cu from Ni in sulphate solutions has been described⁷⁶ using 20% v/v LIX® 984N, an oxime based extractant, in kerosene. The presence of kerosene is to reduce the viscosity of the extractant.
Batch extraction of Cu from chloride solutions has been described⁷⁷ using Cyanex 923, a mixture of four trialkylphosphine oxides, in kerosene. The study notes that the presence of Ni does not have any significant adverse effect on the extraction; it is not stated whether there is any selectivity demonstrated between these metal ions.
Batch extraction of Cu from sulphate solutions has been described⁷⁷ using 1,1,1-trifluoro-2,4-pentanedione (TFA) in an ionic liquid, with sodium sulphate added to facilitate phase separation using a centrifuge, followed by stripping with supercritical CO₂.
In the context of water purification, batch extraction of Cu(II) ions from sulphate and nitrate solutions has been described⁷⁹, using di-2-ethylhexyl phosphoric acid in chloroform.

Other methods of separation of various metal ions from one another are disclosed, which do not concern the metal ions of interest in the present invention³⁷⁻⁴⁵.

All of these methods are batch separations requiring manual input from personnel, and so are capable of improvement from the point of view of safety. The authors of the above separation procedures, except for Dejesus³⁴, do not address the efficiency of the overall process. None of the above procedures consider the reuse of target material where radiometal ions are used.

The use of an automated separation system for the mixed liquid-liquid extraction/resin based separation of ⁹⁹Tc from Mo ion comprised in aqueous solution has been described⁴⁶, in which the use of the column in the separation is automated. This does not of course address the need for manual handling of radiometal solutions in the liquid-liquid extraction batch processes.

One of the important stages of liquid-liquid extraction is the phase separation stage, ie the stage at which the organic extractant and the aqueous phases are separated from one another following their mixing to allow partition of the solutes of the aqueous phase between the aqueous phase and the organic extractant. Traditionally, this has been carried out using such apparatus as a separatory funnel, in which a more dense phase and a less dense phase separate into individual layers and are allowed to flow out of the separatory funnel in turn. Recently, however, techniques for carrying out this stage using a microfiltration membrane have been described^{47,48}. In these procedures, the basis of the phase separation is not density, as in the traditional methods, but interfacial tension between the phases. If the combination of the interfacial tension between the organic and aqueous phases and the membrane is appropriately selected, one of the phases will wet the membrane and the other will not, allowing one phase selectively to pass through the membrane. In this way, the membrane acts as a selective barrier between the phases. Pressure is applied to drive the liquid through the membrane, though again this must be carefully selected with reference to the interfacial tension of the phases and the membrane used in order that the phase separation is completely selective. This method of membrane based phase separation is distinct from extraction methods in which the membrane itself comprises the extractant phase, such as fibre-supported liquid extraction⁴⁹, bulk liquid membranes⁵⁰, emulsion liquid membranes, supported liquid membranes, polymer inclusion membranes and the like⁵¹.

### SUMMARY OF THE INVENTION

It is an aim of the present invention to provide a method for the efficient on demand production of radioisotopes, particularly for medical and veterinary use.

It is an aim of the present invention to provide a means for the efficient separation of a radiometal ion from a target metal ion that minimises manual handling of the radioisotope and target material, to improve safety.

It is an aim of the present invention to provide a method for the production of radioisotopes that minimises manual handling of the radioisotopes to improve safety.

It is an aim of the present invention to provide a method of on demand production of radioisotopes, particularly for medical and veterinary use, in which the use of ⁶⁸Ge isotopes produced by large particle accelerators is avoided.

It is an aim of the present invention to provide a method of on demand production of radioisotopes, particularly for medical and veterinary use, in which the entire process from generation of the radioisotope to obtaining a pure solution of the radioisotope suitable for synthesis of a radiotracer or radiopharmaceutical can be carried out as a continuous process with minimal manual intervention.

It is an aim of the present invention to provide a method of on demand production of radioisotopes, particularly for medical and veterinary use, in which the target material from which the radioisotope is generated can be reused to generate further radioisotope therefrom.

It is an aim of the present invention to provide a method of on demand production of radioisotopes, particularly for medical and veterinary use, in which a precise dose of a radiotracer or radiopharmaceutical can be supplied for a given patient, subject or purpose.

It is an aim of the present invention to provide apparatus suitable to carry out a method addressing one or more of the above aims.

Accordingly, in a first aspect, the present invention provides a method of separation of a radiometal ion from a target metal ion, comprising a first liquid-liquid extraction step in which an organic phase comprising an extractant and an interfacial tension modifier is mixed with an aqueous phase comprising the radiometal ion and the target metal ion in order that the radiometal ion is at least partially transferred to the organic phase, followed by a first phase separation step, wherein the phase separation is carried out in flow comprising the use of a microfiltration membrane to separate the phases based on the interfacial tension between the phases such that a permeate phase passes through the membrane and a retentate phase does not, wherein:
a. the radiometal ion is a ⁶⁸Ga ion, the target metal ion is a ⁶⁸Zn ion, the extractant is selected from one or more dialkyl ethers R¹OR², wherein the two alkyl groups R¹ and R² can be the same or different, or can together form a cyclic ether, and can optionally be substituted, and the interfacial tension modifier is selected from one or more aromatic hydrocarbons, which may optionally be halogenated, and/or one or more C2-C9 alkanes, which may optionally be halogenated; or
b. the radiometal ion is a ⁸⁹Zr ion, the target metal ion is a ^{nat}Y ion, the extractant is a solvent able to function as a bidentate ligand for ⁸⁹Zr via two oxygen atoms, and the interfacial tension modifier is a solvent having similar properties to the extractant, but that are not able to function as a bidentate ligand for the ⁸⁹Zr ion, such that it does not interfere with the ability of the extractant to interact with the ⁸⁹Zr ions; or
c. the radiometal ion is a ⁴⁵Ti ion, the target metal ion is a ^{nat}Sc ion, the extractant is a solvent able to function as a bidentate ligand for ⁴⁵Ti via two oxygen atoms, and the interfacial tension modifier is a solvent having similar properties to the extractant, but that is not able to function as a bidentate ligand for the ⁴⁵Ti ion, such that it does not interfere with the ability of the extractant to interact with the ⁴⁵Ti ions; or
d. the radiometal ion is a ⁶⁴Cu ion, the target metal ion is a ⁶⁴Ni ion, the extractant is selected from: one or more trialkyl phosphine oxides; one or more alkylphosphoric acid monoalkyl esters; one or more diketones having the structure R³—C(═O)CH₂C(═O)—R⁴, in which R³ and R⁴ are each independently an alkyl or an aryl group; and one or more aldoximes or ketoximes in which the substituent(s) of the oxime group are aromatic groups; and the interfacial tension modifier is a solvent comprising one or more straight or branched chain cyclic or acyclic aliphatic alkanes having from five to sixteen carbon atoms, and which may optionally be substituted, and/or a solvent comprising one or more aromatic hydrocarbons, which may optionally be substituted.

Preferably, in the method, a pressure ΔPₘₑₘ is exerted across the microfiltration membrane by a pressure controller. Preferably, the pressure exerted across the microfiltration membrane, ΔPₘₑₘ, is controlled to be less than the capillary pressure P_{cap} associated with the fluid passageways of the microfiltration membrane and the mixture of the aqueous phase and the organic phase, and is controlled to be greater than the pressure Pₚₑᵣ required to cause the permeate phase to pass through the microfiltration membrane.

Preferably, the microfiltration membrane is hydrophobic, and the permeate phase is the organic phase. Suitably, however, a hydrophilic microfiltration membrane may be used, in which case the permeate phase will be the aqueous phase.

Preferably, the first liquid-liquid extraction step is conducted in flow. Preferably, the first liquid-liquid extraction step comprises mixing the aqueous phase and the organic phase such that stable liquid-liquid segmented flow of the mixture is established.

Preferably, the aqueous phase is an aqueous solution of a protic acid. Suitably, the aqueous phase comprises a concentration of aqueous hydrochloric acid or nitric acid of greater than or equal to 1 M, such as greater than or equal to 3M, preferably greater than or equal to 6M, and, in some embodiments, most preferably comprises a concentration of 12M aqueous hydrochloric acid or nitric acid. Suitably, the aqueous phase has a pH of less than or equal to 1. Suitably, the aqueous phase is an aqueous solution of nitric acid. Preferably, the aqueous phase is an aqueous solution of hydrochloric acid.

Preferably, the radiometal ion and the target metal ion are defined as follows:
a. the radiometal ion is a ⁶⁸Ga(III) ion and the target metal ion is a ⁶⁸Zn(II) ion; or
b. the radiometal ion is a ⁸⁹Zr(IV) ion and the target metal ion is a ^{nat}Y(III) ion; or
c. the radiometal ion is a ⁴⁵Ti(IV) ion and the target metal ion is a ^{nat}Sc(III) ion; or
d. the radiometal ion is a ⁶⁴Cu(II) ion and the target metal ion is a ⁶⁴Ni(II) ion.

In some embodiments, the radiometal ion is a Ti ion and the target metal ion is a Sc ion. In those embodiments, preferably the aqueous phase is a solution in 12M HCl. Preferably, the extractant is a solvent having the ability to function as a bidentate ligand for Ti via two oxygen atoms, preferably thus forming a five membered ring, as well as having a suitable interfacial tension with 12 M (37%) HCl. Suitable extractants may be maltol, vanillin, eugenol, and guaiacol (o-methoxyphenol). Suitable interfacial tension modifiers are solvents having similar properties to the extractant, but that are not able to function as a bidentate ligand for the Ti ion, such that it does not interfere with the ability of the extractant to interact with the Ti ions, such as fluorobenzene, trifluorotoluene, thiophene and anisole. Preferably, the extractant is guaiacol and the interfacial tension modifier is anisole. More preferably, the anisole is present in an amount of at least 10% v/v. Preferably, the flow ratio of the aqueous phase to the organic phase is 1 to greater than or equal to 3. Preferably, the microfiltration membrane is a PTFE membrane. Preferably, a pressure controller is present in the form of a PFA diaphragm. Most preferably, the microfiltration membrane is a PTFE membrane having a pore size of 0.2 μm, the PFA diaphragm has a thickness of 0.002″ (0.0508 mm). Suitably, the combined flow rate of the organic phase and aqueous phase may be selected in the range of 0.01 mL/min to 12 mL/min, such as 0.1 mL/min to 10 mL/min, or 0.2 mL/min to 8 mL/min, or 0.2 mL/min to 5 mL/min, or 0.2 mL/min to 2 mL/min, such as 0.5 mL/min or 1.00 mL/min.

In some embodiments, the radiometal ion is a Ga ion and the target metal ion is a Zn ion. In those embodiments, preferably the extractant is selected from the group consisting of diethylether, butylmethyl ether, diisopropyl ether, tetrahydropyran, methyl hexyl ether, dibutyl ether and diamyl ether. In some embodiments, the extractant is selected from the group consisting of diethylether, butylmethyl ether, tetrahydropyran, methyl hexyl ether, and dibutyl ether. In some embodiments, the extractant is selected from the group consisting of butylmethyl ether, tetrahydropyran, methyl hexyl ether, and dibutyl ether. More preferably the extractant is selected from butyl methyl ether, diisopropyl ether, dibutyl ether and diethyl ether, yet more preferably the extractant is selected from butyl methyl ether and diisopropyl ether, and most preferably the extractant is diisopropyl ether. In some embodiments, the extractant is not diisopropyl ether, and/or is not diethyl ether. Preferably, the interfacial tension modifier is selected from the group consisting of: a fluorinated aromatic hydrocarbon; an aromatic hydrocarbon; an alkoxybenzene; a halogenated alkane, for example selected from the group consisting of 1,2-dichloroethane, 1,1,2-trichloroethane, 1,1,1-trichloroethane, hexachloroethane and bromoethane; and an alkane; more preferably, the interfacial tension modifier is selected from the group consisting of toluene, anisole, 1,2-dichloroethane, trifluorotoluene and heptane. Yet more preferably, the interfacial tension modifier is selected from the group consisting of toluene and trifluorotoluene, and most preferably the interfacial tension modifier is trifluorotoluene. Preferably, the ratio of the extractant to the interfacial tension modifier is 1:2 by volume. Preferably, the aqueous phase is a solution in 6M HCl. Where this is so, the extractant is preferably selected from diethyl ether, diisopropyl ether, dibutyl ether, butyl methyl ether and hexyl methyl ether, more preferably from diethyl ether, diisopropyl ether and hexyl methyl ether. Equally preferably, the aqueous phase is a solution in 3M HCl. Where this is so, the extractant is preferably selected from diethyl ether and diisopropyl ether, more preferably diisopropyl ether. The conditions under which the separation is carried out may include a concentration of zinc salt, such as ZnCl₂, of more than 5 m, such as 7 m, where m indicates molality (moles of solute per kg solvent).

Where the radiometal ion is a Ga ion and the target metal ion is a Zn ion, it is useful for the method to further comprise a back extraction procedure, in order that the Ga ion can be brought back into the aqueous phase for use in radiolabeling reactions. This back extraction procedure comprises, following the first phase separation step, a first back-extraction step in which an organic phase comprising the radiometal ion is mixed with an aqueous solution of a protic acid in order that the radiometal ion is at least partially transferred to the aqueous solution, followed by a back-extraction phase separation step, in which the phase separation is carried out in flow comprising the use of a microfiltration membrane to separate the phases based on the interfacial tension between the phases such that a permeate phase passes through the membrane and a retentate phase does not, in order to obtain an aqueous solution comprising the radiometal ion. Preferably, the aqueous solution of a protic acid is an aqueous solution of less than 6 M HCl, such as less than 3 M HCl, more preferably 0.001 to 1 M HCl, most preferably 0.1 M HCl.

Where the radiometal ion is a Ga ion and the target metal ion is a Zn ion, it is useful for the method to further comprise a scrubbing procedure, in order to reduce the quantity of Zn in the organic phase and thus improve the purity with which the Ga is obtained. This scrubbing procedure comprises, following the first phase separation step, a first scrubbing step in which an organic phase comprising the radiometal ion and the target metal ion is mixed with an aqueous solution of a protic acid in order that the target metal ion is at least partially transferred to the aqueous solution, followed by a scrubbing phase separation step, in which the phase separation is carried out in flow comprising the use of a microfiltration membrane to separate the phases based on the interfacial tension between the phases such that a permeate phase passes through the membrane and a retentate phase does not, in order to obtain an aqueous solution comprising the target metal ion, and an organic phase comprising the radiometal ion and a decreased quantity of the target metal ion. Preferably, the aqueous solution of a protic acid is an aqueous solution of at least 8 M HCl.

In order to obtain a high purity of Ga, it is preferable that the method further comprises, following the first liquid-liquid extraction step and the first phase separation step, and in this order: the scrubbing procedure described above; and then a first back extraction procedure as described above. Yet more preferably, the method can further comprise, following the first back extraction procedure: a second liquid-liquid extraction step and a second phase separation step as described above; and then a second back extraction procedure as described above. Preferably, the aqueous solution comprising the radiometal ion obtained from the first back extraction procedure is acidified prior to its introduction into the second liquid-liquid extraction step as the aqueous phase, preferably to a 6N acid concentration.

Preferably, the microfiltration membrane is selected from a PTFE membrane with PP support and a PTFE membrane. Preferably, a pressure controller is present in the form of a PFA diaphragm. Most preferably, the microfiltration membrane is selected from a PTFE membrane with PP support and a PTFE membrane, and has a pore size of 0.2 μm, the PFA diaphragm has a thickness of 0.002″ (0.0508 mm). Suitably, the combined flow rate of the organic phase and aqueous phase may be selected in the range of 0.01 mL/min to 12 mL/min, such as 0.1 mL/min to 10 mL/min, or 0.2 mL/min to 8 mL/min, or 0.2 mL/min to 5 mL/min, or 0.2 mL/min to 2 mL/min, such as 0.5 mL/min or 1.00 mL/min.

In some embodiments, the radiometal ion is a Zr ion and the target metal ion is a Y ion. Preferably, the extractant is a solvent having the ability to function as a bidentate ligand for Zr via two oxygen atoms, preferably thus forming a five membered ring, as well as having a suitable interfacial tension with 12 M (37%) HCl. Suitable extractants may be maltol, vanillin, eugenol, and guaiacol (o-methoxyphenol). Suitable interfacial tension modifiers are solvents having similar properties to the extractant, but that are not able to function as a bidentate ligand for the Zr ion, such that it does not interfere with the ability of the extractant to interact with the Zr ions, such as fluorobenzene, trifluorotoluene, thiophene and anisole. Preferably, the extractant is guaiacol (o-methoxyphenol), and the interfacial tension modifier is anisole. Preferably, the anisole is present in an amount of at least 10% v/v. Preferably, the aqueous phase is a solution in 12 M HCl. Preferably, the flow ratio of the aqueous phase to the organic phase is 1 to greater than or equal to 3, and more preferably is 1:5. Alternatively, where the radiometal ion is a Zr ion and the target metal ion is a Y ion, it is preferable that the extractant is 0.1 M trioctylphosphine oxide (TOPO), the interfacial tension modifier is hexane, and the aqueous phase is a solution in 6 M HCl.

Preferably, the microfiltration membrane is a PTFE membrane. Preferably, a pressure controller is present in the form of a PFA diaphragm. Most preferably, the microfiltration membrane is a PTFE membrane having a pore size of 0.2 μm, the PFA diaphragm has a thickness of 0.002″ (0.0508 mm). Suitably, the combined flow rate of the organic phase and aqueous phase may be selected in the range of 0.01 mL/min to 12 mL/min, such as 0.1 mL/min to 10 mL/min, or 0.2 mL/min to 8 mL/min, or 0.2 mL/min to 5 mL/min, or 0.2 mL/min to 2 mL/min, such as 0.5 mL/min or 1.00 mL/min.

In some embodiments, the radiometal ion is a Cu ion and the target metal ion is a Ni ion. Preferably, the extractant is a species having the ability to act as a monodentate or bidentate ligand for the Cu ion, as well as having a suitable interfacial tension with 6 M HCl.

Suitable extractants may be: one or more trialkyl phosphine oxides; one or more alkylphosphoric acid monoalkyl esters; one or more diketones having the structure R³—C(═O)CH₂C(═O)—R⁴, in which R³ and R⁴ are each independently an alkyl or an aryl group; and one or more aldoximes or ketoximes in which the substituent(s) of the oxime group are aromatic groups.

Suitable interfacial tension modifiers are solvents, such as branched or unbranched cyclic or acyclic aliphatic hydrocarbons having from five to sixteen carbon atoms, or aromatic hydrocarbons. Preferably, the interfacial tension modifier is selected from n-pentane, n-hexane, n-heptane, n-octane, n-nonane, n-decane, n-undecane, i-hexane, neo-hexane, i-heptane, neo-heptane, cyclohexane, cycloheptane, cyclooctane, kerosene, light petroleum, benzene, naphthalene, toluene, ethylbenzene, dimethylbenzene, iso-octane and mixtures thereof, more preferably selected from toluene, hexane, heptane and mixtures thereof.

Preferably, the extractant is selected from:
one or more trialkylphosphine oxides, in which the alkyl groups are selected from straight chain or branched hydrocarbon chains having from six to ten carbon atoms, such as Cyanex 923 (TRPO) or trioctylphosphine oxide (TOPO);
one or more alkylphosphoric acid monoalkyl esters of the structure R⁵—P(═O)(OH)—OR⁶, where R⁵ and R⁶ are each independently a branched or unbranched C₆ to C₁₀ alkyl group, such as 2-ethylhexyl phosphoric acid mono-2-ethylhexyl ester (PC-88A);
one or more diketones having the structure R³—C(═O)CH₂C(═O)—R⁴, in which R³ and R⁴ each independently are an optionally halogenated branched or unbranched C₁ to C₁₀ alkyl group or a substituted or unsubstituted phenyl group, such as 1-phenyldecane-1,3-dione, heptadecane-8,10-dione, or 1,3-diphenylpropane-1,3-dione;
one or more aldoximes or ketoximes having an aromatic substituent wherein the benzene ring is substituted with both an oxygen and an alkyl group, such as 5-nonylsalicylaldoxime, 5-dodecylsalicylaldoxime, Acorga® P50, or 2-hydroxy-5-nonylacetophenone oxime.

More preferably, the extractant is trioctyl phosphine oxide.

Preferably, the extractant, preferably trioctylphosphine oxide, is present in a concentration of at least 0.1 M in the interfacial tension modifier. Preferably, the extractant, preferably trioctyl phosphine oxide, is present in a concentration of from 0.1 M to 0.4 M in the interfacial tension modifier. Preferably, the extractant is 0.4 M trioctyl phosphine oxide, and the interfacial tension modifier is toluene; alternatively, the extractant is 0.1 M trioctylphosphine oxide and the interfacial tension modifier is hexane or heptane. Preferably, the aqueous phase is a solution in 6 M HCl.

Preferably, the flow ratio of the aqueous phase to the organic phase is 1 to greater than or equal to 1, more preferably, 1 to greater than or equal to 3, and most preferably in the range of from 1 to greater than or equal to 3 to 1 to less than or equal to 5.

Preferably, the microfiltration membrane is a PTFE membrane. Preferably, a pressure controller is present in the form of a PFA diaphragm. Most preferably, the microfiltration membrane is a PTFE membrane having a pore size of 0.2 μm, the PFA diaphragm has a thickness of 0.002″ (0.0508 mm). Suitably, the combined flow rate of the organic phase and aqueous phase may be selected in the range of 0.01 mL/min to 12 mL/min, such as 0.1 mL/min to 10 mL/min, or 0.2 mL/min to 8 mL/min, or 0.2 mL/min to 5 mL/min, or 0.2 mL/min to 2 mL/min, such as 0.5 mL/min or 1.00 mL/min.

In a second aspect, the present invention provides a method of generation of radiometal ions from a target metal, comprising:
a. providing an aqueous solution of ions of the target metal;
b. irradiation of the target metal ion solution with a particle beam to produce a mixture of radiometal ions and target metal ions in aqueous solution;
c. separation of the radiometal ions from the target metal ions according to the method of the first aspect of the invention.

Preferably, the method further comprises, following step c, recycling the aqueous solution of the target metal ions for use in a subsequent irradiation step b. Suitably, the recycling of the aqueous solution of the target metal ions comprises the step of treating the aqueous solution of the target metal ions to remove any organic solvents from the solution. Preferably, the treatment step comprises passage of the aqueous solution of the target metal ions through a reverse phase chromatography column having a stationary phase suitable for adsorption of any trace organic solvents. Preferably, the reverse phase chromatography column is a C18 column, ie an octadecyl carbon chain bonded silica stationary phase column.

Suitably, the irradiation is conducted using a cyclotron. Suitably, when irradiating Zn to produce ⁶⁸Ga, or Sc to produce ⁴⁵Ti, the irradiation may comprise bombardment of the target metal ion solution with protons having an energy of 12-13 MeV, preferably 12.5 MeV, for example at a current of 5 μA for 5 to 20 min (Zn) or at a current of 10-20 μA for 5-15 min (Sc). For irradiation of Y to produce ⁸⁹Zr, the irradiation may comprise bombardment of the target metal ion solution with protons having an energy of 12.5-15 MeV, for example at a current of 25 μA for around 5 min. For irradiation of ⁶⁴Ni to produce ⁶⁴Cu, the irradiation may comprise irradiation with 11 MeV protons using 20 μA current for 360 min. The mixture of radiometal ions and target metal ions in aqueous solution may comprise a concentration of target metal ion salt, such as chloride salt, of at least 0.1 M, preferably 1 M, or preferably more than 5 m, such as 7 m.

In a third aspect, the present invention provides a method of generation of radiometal ions from a target metal, comprising:
a. providing a solid target metal;
b. irradiation of the solid target metal with a particle beam to produce a solid mixture of radiometal and target metal;
c. dissolution of the solid mixture of radiometal and target metal to produce an aqueous solution comprising radiometal ions and target metal ions;
d. separation of the radiometal ions from the target metal ions according to the method of the first aspect of the invention.

Suitably, the irradiation is conducted using a cyclotron. Suitably, when irradiating Zn to produce ⁶⁸Ga, or Sc to produce ⁴⁵Ti, the irradiation may comprise bombardment of the target metal with protons having an energy of 12-13 MeV, preferably 12.8 MeV, for example at a current of 10 μA for 160 min (Zn) or at a current of 10-20 μA for 5-15 min (Sc). For irradiation of Y to produce ⁸⁹Zr, the irradiation may comprise bombardment of the target metal with protons having an energy of 13.1 MeV, for example at a current of 25 μA for around 5 min. For irradiation of ⁶⁴Ni to produce ⁶⁴Cu, the irradiation may comprise irradiation with 11 MeV protons using 20 μA current for 360 min.

Suitably, Ni is irradiated in the form of an electroplated layer. Suitably, the irradiated Ni (the solid mixture of radiometal and target metal) is dissolved in a 30% HCl for 30 min at 60° C., and additional 5 minutes at 80° C., then diluted to 6M HCl. Suitably, Sc, Y and Zn are each irradiated in the form of a metal foil. Suitably, the irradiated Sc and Y foils (the solid mixture of radiometal and target metal) are dissolved in 30-37%12M HCl at ambient temperature for a sufficient time to dissolve the foil, usually a few minutes, then diluted to 12M HCl. Suitably, the irradiated Zn foils (the solid mixture of radiometal and target metal) are dissolved in 3 M or 6 M HCl at ambient temperature for a sufficient time to dissolve the foil, usually a few minutes.

These solutions can be directly used for the liquid-liquid extraction.

In this aspect, the aqueous solution containing target metal ions resulting from the liquid-liquid extraction cannot be directly recycled for further irradiation as it is not a solid metal foil, though it can be recycled by use in a process according to the second aspect of the invention. Preferably, if the said solution is to be used in a process according to the second aspect of the invention, it is first subjected to a step of treatment to remove any organic solvents from the solution. Preferably, the treatment step comprises passage of the aqueous solution of the target metal ions through a reverse phase chromatography column having a stationary phase suitable for adsorption of any trace organic solvents. Preferably, the reverse phase chromatography column is a C18 column, ie an octadecyl carbon chain bonded silica stationary phase column.

In a fourth aspect, the present invention provides a method of production of a radiolabelled pharmaceutical, wherein the radiometal used in the radiolabeling is selected from ⁴⁵Ti and ⁸⁹Zr, comprising the method of the second or the third aspect of the invention, followed by the step of reaction of the solution of separated radiometal ions resulting from step c with a reactive precursor of the radiolabelled pharmaceutical. Reaction protocols for the production of radiolabeled pharmaceuticals are well known to the skilled person. For example, the synthesis of an ⁸⁹Zr containing radiotracer has been described²⁷, in which ⁸⁹Zr in a 1 M HEPES buffer is pH adjusted to within the range 6.8-7.2 with 2M sodium hydroxide or 2M hydrochloric acid. Trastuzumab-DFO (10 mg/mL) is added to the solution to create the reaction solution, which is pumped through a single channel reactor at a total flow rate of 20 μL/min, optionally followed by incubation at 37° C. for 1 h by halting the flow. The product, ⁸⁹Zr-Trastuzumab, is collected in a microcentrifuge tube and the radiochemical yield confirmed by instant TLC. A method of producing a ⁴⁵Ti containing radiotracer is described¹⁸ and is reproduced in Example 4. Copper radionuclide containing radiopharmaceuticals, such as Cu-ASTM used in imaging hypoxic tissues, have been described⁷⁵.

In a fifth aspect, the present invention provides a method of production of a radiolabelled pharmaceutical, wherein the radiometal used in the radiolabeling is ⁶⁸Ga, comprising the method of the second or the third aspect of the invention, and further comprising:

a back extraction procedure as described above in the first aspect of the invention, in which the organic phase is that resulting from the separation step of the second or third aspect of the invention; followed by
reaction of the aqueous solution resulting from the back extraction procedure with a reactive precursor of the radiolabelled pharmaceutical.

Reaction protocols for the production of radiolabeled pharmaceuticals are well known to the skilled person. For example, the synthesis of a ⁶⁸Ga-containing radiotracer has been described,⁸ in which 40 μl of an aqueous solution of ⁶⁸Ga is added to a PSMA conjugate (0.1-1 nmol in 0.1 M HEPES buffer, pH 7.5, 100 μl) and 10 μl HEPES buffer (2.1 M in H₂O). The pH of the solution is adjusted using NaOH. The reaction mixture is incubated at room temperature or 95° C., depending on the conjugate used. A ⁶⁸Ga-PSMA radiotracer is produced.

In a sixth aspect, the present invention provides the use of phase separation in flow, comprising the use of a microfiltration membrane to separate an organic phase from an aqueous phase based on the interfacial tension between the phases such that a permeate phase passes through the membrane and a retentate phase does not, in the liquid-liquid extraction of a radiometal ion from a target metal ion. Features mentioned in connection with the first aspect of the invention are also relevant to the sixth aspect of the invention.

In a seventh aspect, the present invention provides apparatus for conducting separation of a radiometal ion from a target metal ion by means of a liquid-liquid extraction and phase separation carried out in continuous flow, the phase separation being preferably according to the first aspect of the invention, comprising:

a first inlet for an aqueous phase comprising the radiometal ion and the target metal ion;
a second inlet for an organic phase comprising an extractant and an interfacial tension modifier;
one or more mixers for mixing the organic phase and the aqueous phase;
tubing to convey the mixture of the organic phase and the aqueous phase;
a phase separation apparatus comprising a microfiltration membrane to separate the organic phase from the aqueous phase based on the interfacial tension between the phases such that a permeate phase passes through the membrane and a retentate phase does not;
a first outlet for the aqueous phase exiting the phase separation apparatus;
a second outlet for the organic phase exiting the phase separation apparatus.

Preferably, the phase separation apparatus further comprises a pressure controller to control the pressure ΔPₘₑₘ exerted across the microfiltration membrane.

Preferably, the pressure controller is in the form of a diaphragm. Suitably, the diaphragm is made of a polymer selected from the group consisting of perfluoroalkoxyalkane (PFA), latex, polytetrafluoroethylene (PTFE), fluorinated ethylene propylene (FEP), fluoroelastomers (FMK), perfluoroelastomers (FFKM), tetrafluoro ethylene/polypropylene rubbers (FEPM), neoprene, nitrile rubber, and polyethylene. Preferably, the diaphragm is made of perfluoroalkoxyalkane (PFA). Preferably, the diaphragm thickness is 0.002″ (0.0508 mm).

Suitably, the microfiltration membrane is made from a polymer selected from the group consisting of polytetrafluoroethylene (PTFE), polyvinylidene fluoride (PVDF), cellulose acetate, polysulfane, polysulfone, polyethersulfone, polypropylene, polyethylene, and polyvinyl chloride (PVC). Preferably, the microfiltration membrane is made from polytetrafluoroethylene (PTFE). Suitably, the microfiltration membrane has a pore size selected in the range 0.1 to 1.0 μm, such as 0.1 to 0.5 μm, or 0.2 to 0.5 μm. Preferably, the microfiltration membrane has a pore size of 0.2 μm.

Suitably, the one or more mixers comprise mixers selected from the group consisting of: Y-junction mixing tees; T-junction mixing tees; static mixers; packed beds containing sand, stainless steel beads or glass beads; or combinations thereof. Preferably, the one or more mixers are one T-junction mixing tee and two static mixers. Preferably, the T-junction mixing tee is made of polyethyletherketone (PEEK) and the static mixers are made of polytetrafluoroethylene (PTFE).

Preferably, the apparatus further comprises a mixing loop between the one or more mixers and the phase separation apparatus. Preferably, the mixing loop is made of PFA tubing. Preferably, the mixing loop is 108 cm long.

Preferably, the tubing used in the apparatus is PFA tubing.

Preferably, the apparatus further comprises a pump, such as a syringe pump, upstream of each of the first inlet and the second inlet to drive the aqueous phase and the organic phase, respectively, therethrough.

In an eighth aspect, the present invention provides apparatus for conducting separation of a radiometal ion from a target metal ion, particularly the separation of ⁶⁸Ga from Zn, by means of a liquid-liquid extraction carried out in continuous flow, followed by back-extraction of the radiometal ion, comprising:

a first apparatus according to the seventh aspect of the invention for conducting the liquid liquid extraction;
a second apparatus according to the seventh aspect of the invention for conducting back extraction of the radiometal ion, in which the first inlet is for an aqueous phase for back-extraction of the radiometal ion, and the second inlet is for the organic phase containing the radiometal ion obtained from the first apparatus. Suitably, the second outlet of the first apparatus is connected directly or indirectly to the second inlet of the second apparatus.

Preferably, the apparatus further comprises a third apparatus according to the seventh aspect of the invention for conducting a second liquid liquid extraction; and a fourth apparatus according to the seventh aspect of the invention for conducting a second back extraction of the radiometal ion, in which the first inlet is for an aqueous phase for the second back-extraction of the radiometal ion, and the second inlet is for the organic phase containing the radiometal ion obtained from the fourth apparatus. Suitably, the first outlet of the second apparatus is connected directly or indirectly to the first inlet of the third apparatus. Suitably, the second outlet of the third apparatus is connected directly or indirectly to the second inlet of the fourth apparatus.

In a ninth aspect of the invention, the present invention provides apparatus for conducting separation of a radiometal ion from a target metal ion, particularly the separation of ⁶⁸Ga from Zn, by means of a liquid-liquid extraction carried out in continuous flow, followed by scrubbing of target metal ion from the organic phase, and then back-extraction of the radiometal ion, comprising:

a first apparatus according to the seventh aspect of the invention for conducting the liquid liquid extraction;
a second apparatus according to the seventh aspect of the invention for conducting scrubbing of the organic phase exiting the first apparatus, in which the first inlet is for an aqueous phase for scrubbing the organic phase, and the second inlet is for the organic phase containing the radiometal ion obtained from the first apparatus;
a third apparatus according to the seventh aspect of the invention for conducting back extraction of the radiometal ion, in which the first inlet is for an aqueous phase for back-extraction of the radiometal ion, and the second inlet is for the organic phase containing the radiometal ion obtained from the second apparatus. Suitably, the second outlet of the first apparatus is connected directly or indirectly to the second inlet of the second apparatus. Suitably, the second outlet of the second apparatus is connected directly or indirectly to the second inlet of the third apparatus.

Preferably, the apparatus further comprises a fourth apparatus according to the seventh aspect of the invention for conducting a second liquid liquid extraction; a fifth apparatus according to the seventh aspect of the invention for conducting a second back extraction of the radiometal ion, in which the first inlet is for an aqueous phase for the second back-extraction of the radiometal ion, and the second inlet is for the organic phase containing the radiometal ion obtained from the fourth apparatus. Suitably, the first outlet of the third apparatus is connected directly or indirectly to the first inlet of the fourth apparatus. Suitably, the second outlet of the fourth apparatus is connected directly or indirectly to the second inlet of the fifth apparatus. Preferably, the apparatus further comprises means for acidification of the aqueous phase between the first outlet of the third apparatus and the first inlet of the fourth apparatus.

In each of the eighth and ninth aspects of the invention, the preferred features of each apparatus are as recited for the seventh aspect of the invention. The features and preferred features for each apparatus may be the same or different; preferably, each apparatus is the same.

In a tenth aspect, the present invention provides apparatus for on-demand production of a radiometal from a target metal, comprising:

apparatus for irradiation of a target metal;
apparatus for separation of the radiometal from the target metal according to any one of the seventh to ninth aspects of the invention.

Suitably, the apparatus for irradiation of a target metal comprises a cyclotron, such as a GE PETTrace PT800 cyclotron. Suitably, the apparatus for irradiation of a target metal further comprises means for cooling the target metal, such as direct water cooling. Where the target metal is provided in solution, the apparatus comprises a liquid target chamber, preferably made of niobium, such as a GE PETTrace liquid target chamber.

In an eleventh aspect, the present invention provides apparatus for on-demand production of a radiolabeled compound, comprising:

apparatus for irradiation of a target metal;
apparatus for separation of the radiometal from the target metal according to any one of the seventh to ninth aspects of the invention;
apparatus for reaction of the radiometal solution obtained from the separation step with a reactive precursor of the radiolabeled compound.

Suitably, the apparatus for irradiation of a target metal comprises a cyclotron, such as a GE PETTrace PT800 cyclotron. Suitably, the apparatus for irradiation of a target metal further comprises means for cooling the target metal, such as direct water cooling. Where the target metal is provided in solution, the apparatus comprises a liquid target chamber, preferably made of niobium, such as a GE PETTrace liquid target chamber.

Suitably, the apparatus for reaction of the radiometal solution comprises continuous flow reaction apparatus, such as has been widely described in the literature⁴⁷. For example, the apparatus may comprise, in suitable combinations for the reaction to be carried out: pumps, such as syringe pumps; mixers, such as Y-junction mixing tees, T-junction mixing tees, static mixers, packed beds containing sand, stainless steel beads or glass beads; mixing and/or reaction loops of tubing of suitable length for the reaction process; heating and/or cooling apparatus such as water, ice or oil baths through which the reaction tubing passes.

### BRIEF DESCRIPTION OF THE DRAWINGS

FIG. 1 is a schematic diagram of the general apparatus used to conduct liquid-liquid extraction in flow (LLEF module).

FIG. 2 is a schematic diagram of the setup used for continuous phase separation. The aqueous and the organic phases were combined through a tee and mixed with two static mixers and mixing tubing. The aqueous phase was retained by the membrane, while the organic phase permeated through the membrane. Ti was selectively extracted over Sc into the organic phase.

FIG. 3 is a graph depicting extraction performance for Ti/Sc against time for a total flow rate of 0.20 mL/min (solid symbols) and for a five-fold scale up at a flow rate of 1.00 mL/min (open symbols).

FIG. 4 is a graph depicting extraction performance for Ti/Sc for different residence times in the apparatus. The maximum Ti extraction (90%) was achieved for all residence times, down to the shortest residence time of 13.7 s.

FIG. 5 is a graph depicting extraction efficiency for Ti/Sc against time for a flow rate of 1:3 aqueous:organic with 90% guaiacol 10% anisole. ⁴⁵Ti extraction efficiency was calculated from the radioactivity measurements and Sc extraction calculated from ICP-AES.

FIG. 6 is a radio-HPLC trace of [⁴⁵Ti] (salan)Ti(dipic): FIG. 6A is the HPLC trace for (salan)Ti(dipic), retention time 11.2 min, and FIG. 6B is the radio-TLC trace for [⁴⁵Ti] (salan)Ti(dipic) Rf 0.49 (red peak) and baseline (green peak).

FIG. 7 is a graph showing the extraction percentage for Zr against time for LLEF of Zr from 0.01 M ZrCl₄ solution in 37% HCl, also containing 0.01 M YCl₃, using the guaiacol/anisole, 9/1 v/v mixtures and 1/3 and 1/5 aq/org ratios. Low flow: 0.05/0.15 mL min, aq/org at 1/3 aq/org, and 0.033/0.1667 mL/min at 1/5 aq/org; high flow: 0.25/0.75 mL min. aq/org at 1/3 aq/org.

FIG. 8 is a graph showing the extraction percentage for ⁸⁹Zr against time at low flow rates for LLEF of ⁸⁹Zr from its solution in 37% HCl, also containing 0.01 M YCl₃, resulting from the irradiation of yttrium foil followed by dissolution in 37% HCl, at low flow rates (0.033/0.166 mL/min, aq/org) using the guaiacol/anisole, 9/1 v/v mixture.

FIGS. 9A and 9B are graphs showing the effect of adding an interfacial tension modifier to phase separation in a mixture of a dialkyl ether and hydrochloric acid also containing 7m zinc chloride.

FIG. 10 is a schematic diagram of the apparatus used to carry out LLEF to separate Ga and Zn using one of a selection of dialkyl ethers combined with TFT, and hydrochloric acid, also containing 7 m zinc chloride, followed by back-extraction of Ga into aqueous solution. The process can be performed stepwise.

FIG. 11 is a graph showing the extraction efficiency for LLEF of Ga and Zn using the mixture of dialkyl ethers, TFT, and hydrochloric acid, also containing 7 m zinc chloride, followed by back-extraction of Ga into 0.1 M HCl

FIG. 12 is a schematic depicting the two stage liquid liquid extraction in flow of Ga and Zn using the mixture of dialkyl ethers, TFT, and hydrochloric acid, also containing 7 m zinc chloride, and including scrubbing of residual Zn with 8 M HCl and back-extraction of Ga into 0.1 M HCl. The process can be performed stepwise.

FIG. 13 is a schematic depicting an apparatus for continuous on-demand production of a radioisotope using the separation method and apparatus of the invention.

FIG. 14 is a graph showing the extraction efficiency for LLEF of Cu and Ni using 0.1 M TOPO in toluene at flow rate ratios of 1:1, 1:3 and 1:5 (aq:org).

FIG. 15 is a graph showing the extraction efficiency for LLEF of Cu using 0.1 M TOPO in heptane in the presence of Ni, Co, Fe, Zn, and Ag.

### DETAILED DESCRIPTION

Apparatus for Separation of Radiometal Ions

As noted above, liquid-liquid extraction (LLE) is a widely used means of separation of components of a solution by partitioning the components between two different solvents. Traditionally, this has been conducted between immiscible solvents which separate under the influence of gravity in such apparatus as a separatory funnel, or which are forced to separate by use of apparatus such as a centrifuge. The extraction is based on the relative solubilities of the components of the solution in the chosen immiscible liquids, usually an aqueous phase and an organic phase. In some cases, the system of organic and aqueous phases plus one or more components may form an emulsion or “third phase” which can prevent or make less effective the partitioning of the components between easily separable organic and aqueous phases.

“Extraction” is used to describe the transfer of a component from the aqueous phase to the organic phase, whereas “stripping” or “back-extraction” describes the transfer of a component of interest from the organic phase to the aqueous phase. Removal of an unwanted component from the organic phase is described as “scrubbing”.

In order that an extraction or back-extraction takes place efficiently, it is necessary for the organic and aqueous phases containing the components to be extracted to be thoroughly mixed, in order to permit partitioning of the components between the phases according to their relative solubilities in the phases, followed by a means of separating the two phases from one another. Traditionally, in a separatory funnel, this would be carried out by shaking the separatory funnel containing the phases and components, followed by allowing the phases to separate under gravity, and running off the phases in turn from the bottom of the funnel. More than one extraction step can be carried out to ensure the maximum extraction of the desired components.

It has been recognized by the present inventors that it would be desirable to avoid the handling of solutions containing radioactive materials by an operator, as would be required by these traditional manual methods of liquid-liquid extraction. Nonetheless, such separations are attractive as they are simple and inexpensive to carry out, compared with other methods such as chromatographic methods that may require expensive media, and may result in the need to dispose of radioactively-contaminated media following use. In addition, liquid-liquid extraction can be conducted on a wide range of scales.

Recently, new methods of, and apparatus for, conducting mixing of fluids and separation of fluids have been developed, which methods may be conducted in a continuous manner. The present inventors have recognized that such apparatus and methods could potentially have applicability in the separation of metal ions, in particular the separation of radiometal ions from target metal ions. As far as the present inventors are aware, no such use has been made of these new methods and apparatus in the field of the present invention.

Continuous flow synthesis apparatus has been developed recently that allows reactions to be carried out in a continuous manner. A review⁵² of transformations that have been carried out in continuous flow systems lists transformations such as hydrogenations and reductions, oxidation, acid or base catalyzed bond-forming reactions, transition metal catalyzed bond-forming reactions, esterification reactions, protection and deprotection reactions, photocatalysis and enzymatic reactions. Particular attention has been paid to the use of reactive and/or toxic, particularly gaseous, reagents, as they can be generated and used in a closed system: for example, cyanogen bromide⁵³, chlorine azide⁵⁴, ethylene gas⁵⁵, (meth)acryloyl chloride⁵⁶, chlorine gas⁵⁷ and diazomethane⁵⁸. The use of such systems in the automated production of drugs has been suggested⁵⁹. Some continuous purifications involving metals have been reported, such as removal of excess ligand from nanoparticles⁶⁰ and extraction of leached copper from a target compound⁶¹. However, so far as the present inventors are aware, such technologies have not been applied to the separation of metal ions from one another.

WO2004/087283 describes systems which may be used for liquid-liquid separations, amongst other uses, and in which the separation is carried out by means of differential wetting of arrays of capillary tubes. For example, a hydrophilic and a hydrophobic liquid may be mixed, and the mixture brought in contact with one or more capillary tubes having a hydrophobic coating. The hydrophobic liquid thus wets the capillary tube and rises up it, whereas the hydrophilic liquid does not wet the capillary tube and does not enter it. In this way, the hydrophobic liquid passes through the array of capillary tubes and is separated from the hydrophilic liquid.

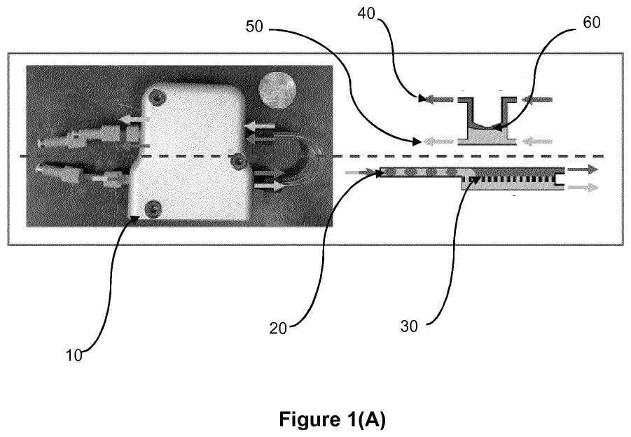

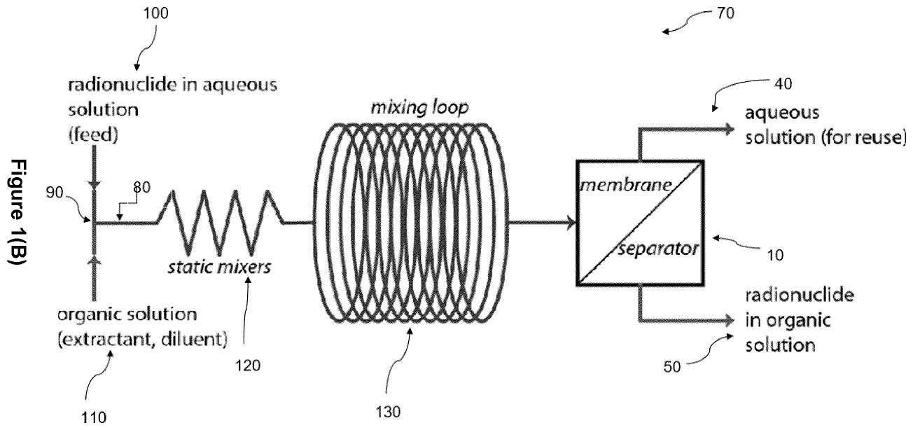

WO2014/026098 describes a membrane separation apparatus suitable for the separation of a first fluid (permeate) from a second fluid (retentate) based on the interfacial tension of the two fluids. In particular, it relates to a system in which a pressure controller is included in the apparatus to apply pressure across the microfiltration membrane that is independent of the pressure downstream of the device, and which can control the selectivity of the membrane for the passage of the fluids, such that one fluid can be allowed to pass selectively through the membrane thus separating it from the other fluid. Such a membrane separation unit is available from Zaiput Flow Technologies. A schematic of such a separator 10 is depicted on the right hand side of FIG. 1A. A mixed phase inlet stream 20 is passed to a microfiltration membrane 30 that divides a retentate outlet stream 40 from a permeate outlet stream 50. As can be seen, the membrane 30 allows the permeate to pass therethrough, but not the retentate. A pressure controller, diaphragm 60, is provided at an interface between the retentate outlet stream 40 and permeate outlet stream 50. A useful practical description of the assembly and use of continuous flow systems containing such membrane separators has recently been published⁴⁷.

Without wishing to be bound by theory⁴⁸, it is believed that, in order that the separation is complete, the capillary pressure P_{cap} associated with the fluid passageways in the membrane and the mixture of fluids to which the membrane is exposed, must not be exceeded, or both fluids will be forced through the membrane. Thus, ΔPₘₑₘ, the pressure difference across the membrane, may not exceed P_{cap}. P_{cap} is quantified as:

$${P}_{cap} = \frac{2 γcosθ}{r}$$

where θ is the contact angle formed between the solid material of the membrane, the first fluid to be separated and the second fluid to be separated, r is the radius of the membrane pores, and y is the interfacial tension with respect to the first fluid to be separated and the second fluid to be separated.

Further, in order to ensure that the whole of the first fluid passes through the membrane, ΔPₘₑₘ must exceed the pressure Pₚₑᵣ needed to cause the permeate liquid to pass through the membrane. Pₚₑᵣ is quantified as:

$${P}_{per} = \frac{8 μ QL}{n π {R}^{4}}$$

where μ is the viscosity of the permeate phase, Q is the entering permeate fluid volumetric flow rate, L is the membrane thickness, n is the number of pores, and R is the pore radius; this assumes that the membrane acts as an array of cylindrical pores.

In addition, the separator must be operated at a flow rate which is suited to the available membrane area; if the flow rate is excessive, both phases may exit both outlets.

Where the pressure drop along the length of the membrane is negligible compared to P_{cap}−Pₚₑᵣ, then ΔPₘₑₘ can be assumed to be constant along the length of the membrane, and the conditions for successful separation are

P_{cap}>ΔPₘₑₘ>Pₚₑᵣ

The first inequality is satisfied by selection of the microfiltration membrane material and pore size in a range appropriate for the separation, and the second by ensuring that the pressure on the retentate side of the membrane is greater than that on the permeate side of the membrane; this additional pressure is provided by the pressure controller. In practice, the actual operating range of pressures is often narrower than the theoretical range given above.

FIG. 1(B) shows a schematic diagram of the apparatus used to conduct liquid-liquid extraction in flow (LLEF module). The apparatus 70 comprises tubing 80 connected to a membrane separator 10. The tubing 80 is connected at an inlet end to a mixing tee 90; the two inlets of the mixing tee are an inlet for aqueous phase 100 and an inlet for organic phase 110. A syringe pump (not shown) is provided upstream of each inlet 100 and 110. Following convergence of these inlets, the mixing tee outlet is connected via tubing 80 to static mixers 120 and subsequently to a variable length mixing loop 130, before connection to the membrane separator 10. Downstream of the membrane separator 10 are the outlets for organic phase 40 and aqueous phase 50. The metal ion of interest is contained in the aqueous phase introduced through inlet 100, and in the organic phase passing through outlet 40. The aqueous phase passing through outlet 50 comprises the target metal ion from which the metal ion of interest has been produced. This outlet may be directed to waste, or may be further processed to recycle the target metal.

While the apparatus for mixing of the two phases is described here as a mixing tee followed by static mixers, it will be appreciated that other combinations of mixing apparatus (either passive or active) can be used depending on the degree of mixing required, the nature of the fluids to be mixed, and the volume of fluid to be mixed. For example: fewer or more static mixers may be employed; the mixing tee may be a Y-junction mixer or a T-junction mixer; a packed bed reactor housing sand, stainless steel or glass beads may replace one or more of the mixers depicted in FIG. 1(B), especially for difficult to mix fluids or larger fluid volumes. However, in the interests of minimising the complexity of the apparatus and the production of radio-contaminated packings requiring careful disposal, the depicted apparatus is preferred. The materials from which the static mixers and mixing tee are made are chosen with reference to their chemical compatibility with the solvent system, and the pressures that they will need to withstand in operation. The present inventors have found that polyethyletherketone (PEEK) is a suitable material for the mixing tee and that polytetrafluoroethylene (PTFE) is a suitable material for the static mixers for the solvent systems used herein.

The variable length mixing loop, along with the other tubing used in the apparatus, is made from a material chosen with reference to its chemical compatibility with the solvent system, and the pressures that it will need to withstand in operation; the present inventors have found that PFA tubing is suitable for use with the solvent systems used herein. The length of the mixing loop, and the other mixers used, are selected in order to ensure an adequate degree of mixing of the phases, and a residence time in the apparatus sufficient to ensure efficient partitioning of the metal ions between the phases, for the chosen solvent system and the metal ions to be separated.

For the separation of radiometals, the total production time is a critical parameter in the system since the radiometal is continuously undergoing decay back to the target metal. Therefore, the shortest possible residence time is desirable, and the mixing of the phases must be optimized to ensure efficient extraction in as short a time as possible.

It has been found by the present inventors that achieving liquid-liquid segmented flow (sometimes referred to as “slug flow”, though this term more usually refers to gas-liquid mixtures) in the mixed fluid stream passing through the tubing 80, mixing loop 130 and on to the membrane separator 10 is of importance in the present invention. Liquid-liquid segmented flow describes a flow pattern through a tube or pipe in which a first fluid is dispersed in a second fluid in the form of segments of varying length. During stable liquid-liquid segmented flow, the first fluid is shed from the back of the segment at the same rate as it is picked up at the front of the segment, and so the segment length remains constant as it travels along the tube. The present inventors have found that the high mass transfer in liquid-liquid segmented flow systems is particularly beneficial in allowing the efficient partition of components between two phases for the purposes of liquid-liquid extraction. Accordingly, the mixers provided in the apparatus are selected such that liquid-liquid segmented flow is provided in the mixing loop 130 for the combination and volume of fluids used. The present inventors have found that, in the solvent systems used herein, liquid-liquid segmented flow is achieved by mixing of the phases through mixing tee 90. When static mixers are used also, the performance of the extraction was further improved.

Liquid-liquid segmented flow may be determined by visual inspection of the mixture as it flows through the tubing, or may be detected for example by a phototransistor device which clips on to the outside of the tubing and detects a phase interface by alteration in current flow depending on the amount of light received. These devices can detect phase interfaces even in mixtures of colourless liquids. Such devices are available from Optek Technology (OPB350 and OCB350 series). These devices can also be used at the outlets of the separator to detect whether retention or breakthrough of a phase has occurred.

As discussed above, the membrane separator 10 comprises two main components: a polymer microfiltration membrane 30 and a thin diaphragm 60 (FIG. 1A).

The diaphragm 60 acts to modulate the pressure between the aqueous and organic sides of the membrane 30. The aqueous phase is retained by the membrane 30, while the organic phase permeates through the membrane 30. The physical properties and geometry of the membrane 30 as well as the chemical nature of the aqueous and organic phases and their interactions with the membrane surface determine the capillary and permeation pressures. The interactions between the aqueous and the organic phases determine the interfacial tension. The interplay between these parameters determines whether the conditions are within the operating range of the system. If they are not, incomplete phase separation will occur.

Pressure control may be provided by controlling the pressure at each of the outlets of the separation apparatus; however, to do so makes it difficult to integrate the apparatus with other downstream components. Accordingly, it is preferable to use a pressure controller, as described in WO2014/026098 and shown in FIG. 1(A), in the form of a diaphragm.

The diaphragm may be made from a polymer selected from the group consisting of perfluoroalkoxyalkane (PFA), latex, polytetrafluoroethylene (PTFE), fluorinated ethylene propylene (FEP), fluoroelastomers (FMK), perfluoroelastomers (FFKM), tetrafluoro ethylene/polypropylene rubbers (FEPM), neoprene, nitrile rubber, and polyethylene. The diaphragm material should be selected primarily with regard to its resistance to the solvent system to be used in the separation: for example, its acid resistance and/or resistance to organic solvents. The elasticity of the diaphragm following long periods of deformation or physical degradation will also be affected by the choice of diaphragm material. As perfluoroalkoxyalkane (PFA) is a very robust material and is mechanically strong, it is the preferred choice of diaphragm material.

The choice of diaphragm thickness is important as this directly affects the pressure exerted on across the membrane; this must be selected in combination with the membrane properties and solvent system to arrive at a functional apparatus for a given separation. Preferably, the diaphragm thickness is 0.002″ (0.0508 mm).

The microfiltration membrane may be made from a polymer selected from the group consisting of polytetrafluoroethylene (PTFE), polyvinylidene fluoride (PVDF), cellulose acetate, polysulfane, polysulfone, polyethersulfone, polypropylene, polyethylene, and polyvinyl chloride (PVC). As well as chemical compatibility with the solvent system to be used in the separation, the membrane material should be selected having regard to the wettability of the material by the organic phase to be used (for hydrophobic materials such as listed above). It has been found by the present inventors that polytetrafluoroethylene (PTFE) fulfils these requirements for the solvent systems used herein.

The choice of membrane pore size affects the upper and lower boundaries of the pressure of the system, as explained in detail above. Each individual membrane will have a range of pore sizes, and so the pore size specified herein is the manufacturer's specification of pore size, which will represent an average value. The pore size must be selected in combination with the membrane material, the diaphragm thickness and the solvent system to arrive at a functional apparatus for a given separation. Suitably, the microfiltration membrane has a pore size selected in the range 0.1 to 1.0 μm, such as 0.1 to 0.5 μm, or 0.2 to 0.5 μm. Preferably, the microfiltration membrane has a pore size of 0.2 μm.

It will be appreciated from the foregoing discussion that it may be possible for a number of different combinations of membrane material, pore size and diaphragm thickness to provide suitable separation conditions for a given solvent system. Similarly, a number of different solvent systems may be separable in an apparatus having a given combination of membrane material, pore size and diaphragm thickness.

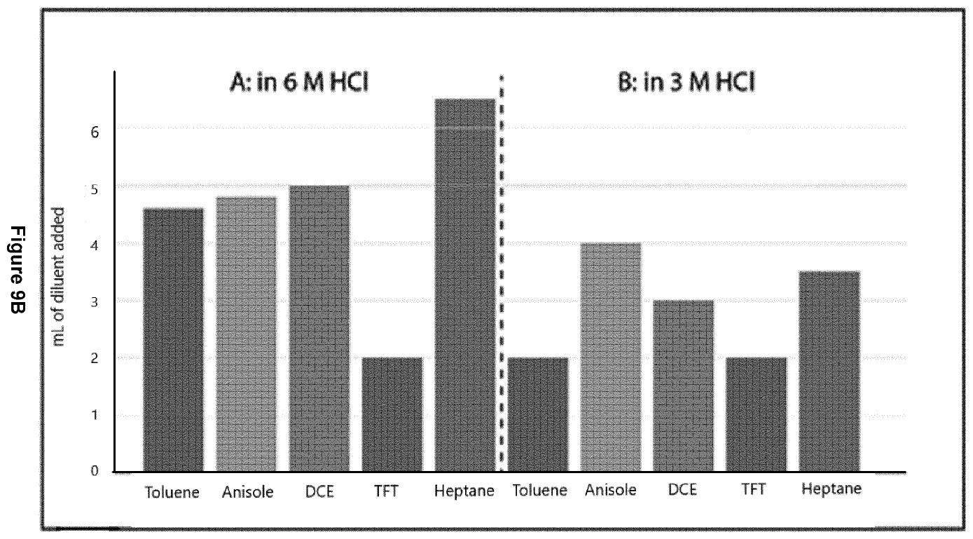

Turning now to FIG. 10, this Figure shows an apparatus 200 for LLEF in which both a liquid-liquid extraction and a subsequent back-extraction (or stripping) step is conducted. This apparatus 200 is suitable for the separation of ⁶⁸Ga from Zn. The liquid-liquid extraction is conducted at the upstream part 270 of the apparatus, and the back-extraction at the downstream part 275. The upstream part 270 is analogous to the LLEF module 70 depicted in FIG. 1B, and comprises: aqueous inlet 100, organic inlet 110, mixing tee 90, tubing 80, static mixers 120, mixing loop 130, membrane separator 10, aqueous outlet 50 and organic outlet 40 (with reference numerals corresponding to those used for corresponding parts of FIG. 1B). However, in this apparatus, the organic outlet 40, containing the metal ion of interest, is connected to the downstream part of the apparatus 275, and is mixed with an aqueous stripping solution followed by a second membrane separation of the phases, in order that the metal ion of interest is back-extracted into the aqueous phase. Thus, the organic phase outlet 50, and second aqueous phase inlet 210 are connected to mixing tee 290, the outlet of which is connected by tubing 280 to static mixers 220, mixing loop 230 and membrane separator 260 in that order. The outlets of the membrane separator are organic phase outlet 240, which is a waste stream, and aqueous phase outlet 250, which contains the metal ion of interest, and which can be subjected to further processing, such as additional purification (for example a second round of LLEF, optionally with a second round of back extraction) and/or a radiolabelling reaction to produce a desired radiolabelled pharmaceutical.

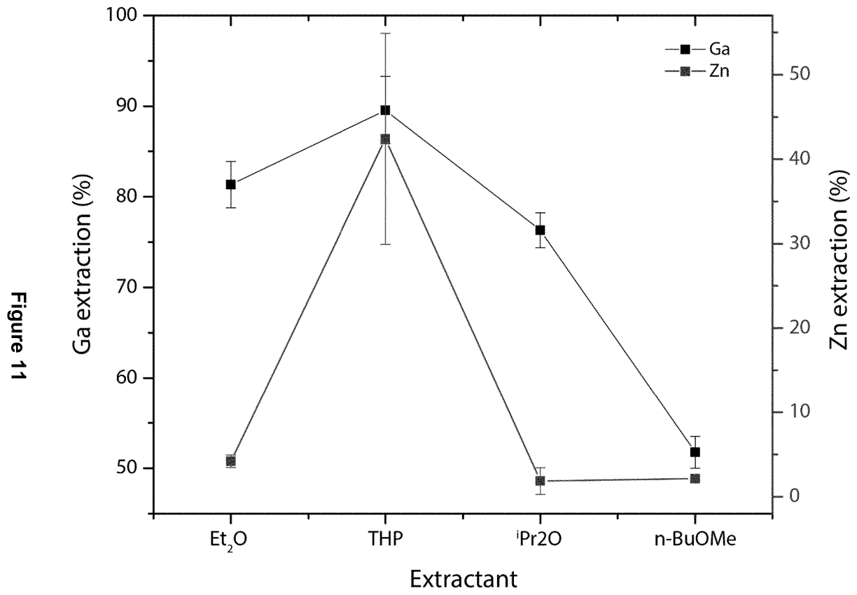

Turning now to FIG. 12, a particularly preferred embodiment 500 of the apparatus of the invention suitable for the separation of ⁶⁸Ga from Zn is depicted. This apparatus permits a first liquid-liquid extraction step to take place at 270, with this section of the apparatus being analogous to that shown at 270 in FIG. 10. Following that, a second extraction at 272 of the organic phase stream 40 against aqueous acid ensures removal of additional Zn from the organic phase stream—this is a scrubbing step. The organic phase stream 350 resulting from this stage passes to a stripping or back extraction step at 275, which is analogous to that shown at 275 in FIG. 10. The aqueous phase stream 250 resulting from this stage is acidified at 410, and then passes to a second liquid-liquid extraction step at 277, and the organic phase stream 420 from this stage is then back-extracted against aqueous acid a second time at 279. The organic phase at organic outlet 440 is a waste solution, and the aqueous phase at aqueous outlet 450 contains the ⁶⁸Ga in acidic aqueous solution, which may be further processed as described above. The second liquid-liquid extraction at 277 and second back extraction are analogous to the first liquid-liquid extraction at 270 and first back extraction at 250 and the apparatus is therefore not further described here.

In the scrubbing stage 272 shown in FIG. 12, the organic phase 40 from the first liquid-liquid extraction, which contains ⁶⁸Ga and some Zn, is fed to mixing tee 390 along with an aqueous acidic solution through aqueous inlet 310. The mixture is passed through tubing 380 to static mixers 320 and mixing loop 330 to partition the metal ions between the aqueous and organic phases. The mixture is then passed to membrane separator 360, and the aqueous phase at aqueous outlet 340 contains Zn ions, and is a waste stream (or recycling stream). The organic phase at organic outlet 350 is passed to the first back extraction step at 275. This additional step reduces the quantity of Zn present in the eventual ⁶⁸Ga aqueous solution.

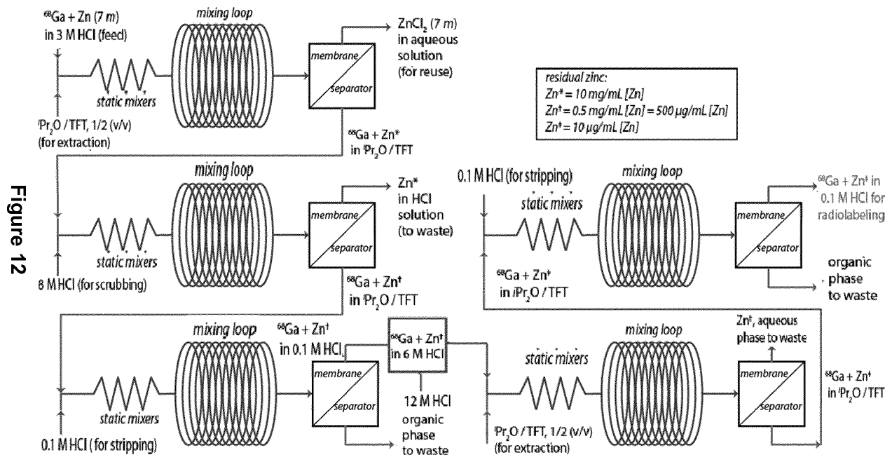

Turning now to FIG. 13, this depicts an apparatus 600 for conducting continuous production of ⁶⁸Ga from Zn. In control room 610, an operator inputs the requested amount of ⁶⁸Ga to be produced. In the cyclotron vault 620, aqueous ⁶⁸ZnCl₂ solution is irradiated at the solution target T. The irradiated target solution is then pumped through the LLEF module 640, as described above with reference to FIG. 1, FIG. 10 or FIG. 12. ZnCl₂ recovered from the LLEF is recycled to the target T. This process is continued until the required quantity of ⁶⁸Ga is obtained as measured by a calibrated radiation detector (not shown) mounted next to the collection vial at the output of S. The desired ⁶⁸GaCl₃ solution is then delivered to a hot cell 630 for radiolabelling. While the process taking place in the apparatus is described for the production of ⁶⁸Ga, it will be appreciated that it is equally applicable to the production of ⁴⁵Ti from a Sc salt in aqueous solution, for production of ⁸⁹Zr from a Y salt in aqueous solution¹⁶, or for production of ⁶⁴Cu from a ⁶⁴Ni salt in aqueous solution.

Separation Methods

The selection of an appropriate extractant to conduct a liquid-liquid extraction in which the phase separation is conducted in flow comprising the use of a microfiltration membrane to separate the phases based on interfacial tension is crucial: the system must provide selective extraction of the metal ion of interest, as little extraction as possible of the target metal ion, must be stable in the presence of the strongly acidic solutions often used in the generation of radiometals (to dissolve irradiated metal foils or irradiated electroplated layers, and/or to avoid the hydrolysis of susceptible metals such as Ti or Zr), and must have a sufficiently high interfacial tension with the aqueous phase that complete separation can be achieved using the microfiltration membrane. This is a much more demanding set of criteria than need be applied to standard batch liquid-liquid separations carried out on the basis of density.

While the present inventors have made reference to literature reports of extraction of particular individual metal ions in batch processes, and in some cases to separation of metal ions from one another in batch processes, it was not expected that these literature conditions would be directly applicable in the processes of the present invention. Indeed, a number of the literature conditions simply did not work at all under the necessary conditions. For example, it had been reported that liquid-liquid batch separation of ⁶⁸Ga ions from ⁶⁸Zn ions in an aqueous solution of protic acid could be carried out using isopropyl ether²⁸. However, under the conditions required for ⁶⁸Ga production (in particular, the presence of around 7 m ZnCl₂ concentration), the phase equilibrium simply did not allow efficient separation; the present inventors found that 65% of the ZnCl₂ present migrated into the organic phase, thus heavily contaminating the ⁶⁸Ga solution with Zn. Other conditions were found to be too inefficient for application in the process of the present invention, for example the use of 1-octanol in the separation of ⁴⁵Ti from Sc, which allowed only 50% extraction efficiency. Yet other extractants attempted by the present inventors did not provide clean phase separation when the microfiltration membrane was applied, but instead led to breakthrough, retention or the formation of emulsions.

It was surprisingly discovered by the present inventors that it is possible to adjust the properties of an extractant with respect to the aqueous phase such that an extractant that did not provide clean phase separation when used alone could do so with the addition of a carefully selected second solvent, here referred to as an interfacial tension modifier. The interfacial tension modifier must not interfere with the interactions between the radiometal ion and the extractant, must not extract the target metal ion to any significant degree, must not dissolve water to any significant extent, and must be able to adjust the properties of the interfacial tension of the overall solvent system (extractant, aqueous phase and interfacial tension modifier (if present)) with respect to the microfiltration membrane such that complete separation of the phases by the microfiltration membrane was possible. The amount of the interfacial tension modifier must be selected carefully to provide optimum separation, as must the relative flow rates of the organic phase (extractant and interfacial tension modifier) and the aqueous phase. The interfacial migration (ie the tendency of one phase to contaminate the other) is a critical parameter which must be minimized to prevent the contamination of aqueous phase with the organic phase, which would make the process incompatible with recycling the target metal solution for a further irradiation step, due to stringent organic-free requirements for aqueous cyclotron solution targets, particularly ZnCl₂.

Thus, for the separation of ⁴⁵Ti from Sc, and for the separation of ⁸⁹Zr from Y, the extractant chosen is a solvent having the ability to function as a bidentate ligand for the radiometal via two oxygen atoms, preferably thus forming a five membered ring, as well as having a suitable interfacial tension with 12 M (37%) HCl. Suitable extractants may be maltol, vanillin, eugenol, and guaiacol (o-methoxyphenol), with guaiacol being particularly preferred. The interfacial tension modifier is a solvent having similar properties to the extractant, though not having the ability to function as a bidentate ligand for the radiometal ion, such that it does not interfere with the ability of the extractant to interact with the radiometal ions, as well as the ability to modify the interfacial tension of the overall system to allow complete separation. Suitable interfacial tension modifiers may be fluorobenzene, trifluorotoluene, thiophene and anisole, with anisole being particularly preferred. For the preferred system of guaiacol and anisole, an amount of anisole of at least 10% v/v is found to perform particularly well, and the optimum flow ratio for the organic phase to the aqueous phase to be greater than 3 to 1, and in some cases 5 to 1. Alternatively, where the radiometal ion is a Zr ion and the target metal ion is a Y ion, it is preferable that the extractant is 0.1 M trioctylphosphine oxide (TOPO), the interfacial tension modifier is hexane, and the aqueous phase is a solution in 6 M HCl.

For the separation of ⁶⁸Ga from Zn, the use of ether extractants was found to work well on combination with an interfacial tension modifier selected from the group consisting of: a fluorinated aromatic hydrocarbon; an aromatic hydrocarbon; an alkoxybenzene; a halogenated alkane, for example selected from the group consisting of 1,2-dichloroethane, 1,1,2-trichloroethane, 1,1,1-trichloroethane, hexachloroethane and bromoethane; and an alkane; particularly, selected from the group consisting of toluene, anisole, 1,2-dichloroethane, trifluorotoluene and heptane, with trifluorotoluene being the most preferred. Preferably, the ratio of the extractant to the interfacial tension modifier is 1:2 by volume, and the optimum flow ratio for the organic phase to the aqueous phase is greater than 3 to 1.

For the separation of ⁶⁴Cu from ⁶⁴Ni, the use of a phosphine oxide extractant was found to work well with an interfacial modifier selected from an aromatic hydrocarbon and an aliphatic alkane which may be cyclic or acyclic; for example selected from the group consisting of n-pentane, n-hexane, n-heptane, n-octane, n-nonane, n-decane, n-undecane, i-hexane, neo-hexane, i-heptane, neo-heptane, cyclohexane, cycloheptane, cyclooctane, kerosene, light petroleum, benzene, naphthalene, toluene, ethylbenzene, dimethylbenzene and iso-octane and mixtures thereof; particularly selected from the group consisting of toluene, heptane and hexane. Preferably, the concentration of the extractant in the interfacial tension modifier is at least 0.1 M, such as from 0.1 M to 0.4 M, and the optimum flow ratio for the organic phase to the aqueous phase is from 5:1 to 3:1.

### EXAMPLES

Separation of Ti and Sc

Materials

Guaiacol (99%), anisole (99%), 1-octanol (99%), titanium (IV) chloride (neat), titanium (IV) chloride solution (0.09 M in 20% HCl), hydrochloric acid (37%), sulfuric acid (95.0-98.0%) and pyridine-2,6-dicarboxylic acid (dipic) (98%) were purchased from Sigma Aldrich and used without further purification. TLC plates (Silica gel on TLC Al foil) were purchased from Sigma Aldrich. Scandium (III) chloride (anhydrous, 99.9%) and scandium foil (250 μm, 99.9% pure, rare earth analysis) were purchased from Alfa Aesar. Custom Ti and Sc ICP standards were purchased from Inorganic Ventures (100 ppm of each metal in a 5% HCl solution). Salan⁶² and (salan)Ti(dipic)³ were synthesized according to the literature procedures.

The membrane separator module was similar to those manufactured by Zaiput Flow Technologies. The aqueous and the organic phases were combined through a tee and mixed with two static mixers and mixing tubing. The aqueous phase was retained by the membrane, while the organic phase permeated through the membrane. Under the conditions of direct extraction, the radionuclide ⁴⁵Ti was selectively extracted from scandium into the organic phase. Pall PTFE membranes were used for all experiments (47 mm diameter, 0.1/0.2/0.5 μm pore size, polypropylene support for the 0.1/0.2 μm pore sizes). Perfluoroalkoxy alkane (PFA) diaphragms (0.001″/0.002″/0.005″ (0.0254/0.0508/0.1270 mm)) were purchased from McMaster Carr. All PFA tubing ( 1/16″ (1.5875 mm) OD, 0.03″ (0.762 mm) ID) was purchased from Idex Health and Science. PTFE static mixers were purchased from Stamixco. The 15 mL plastic centrifuge tubes with screw caps were purchased from VWR.

Radionuclide Production and Separation

For all experiments, the cyclotron target material, scandium, was used at its natural abundance level.

⁴⁵Ti was produced by 10-20 μA proton irradiation of 30-60 mg scandium foil, for 5-15 min using a GE PETtrace cyclotron. To minimize coproduction of the ⁴⁴Ti (half-life=60.0 years), a 500 μm thick aluminium foil was used to degrade the incidental 16 MeV beam to approximately 13 MeV. The irradiated foil was digested in 30-37% M HCl. The mixture was filtered and centrifuged if necessary. The solution was diluted with concentrated hydrochloric acid to make the final dilution ca. 12 M in HCl. These dilutions were used as the aqueous phase for the LLEF.

Instrumentation and Methods

The solutions for the continuous membrane-based separation were pumped using either the KDS 100 Legacy Syringe (radioactive experiments) or the Harvard Apparatus PHD 2000 Programmable and Infusion syringe pumps (non-radioactive experiments). The NMR spectra were taken on Agilent 400 MR operating at 400.445 MHz (¹H). Radio-TLC was performed with a Raytest MiniGita TLC scanner using chloroform/ethyl acetate (1/1, (v/v)) as a mobile phase. The HPLC and radio-HPLC analyses were performed on a Hitachi Chromaster equipped with a Carrol&Ramsey 105-S radio-detector and a Hitachi 5430 double diode array detector. Column: Phenomenex Luna 3μ C18(2) (100 Å, 100 mm×2.00 mm). Flow: 0.5 mL/min. Eluents: (A) 0.1% (v/v) CF₃COOH in Milli-Q water, (B) 0.1% (v/v) CF₃COOH in CH₃CN. The radiochemical identity of [⁴⁵Ti](salan)Ti(dipic) was established by comparing its retention time with that of its natural abundance isotopomer. The radiochemical conversion (RCC) was determined by radio-HPLC or radio-TLC and calculated as:

RCP=(Area_{product}/Total Area)*100%.

The extent of extraction (extraction %) was determined from the radioactivity measurements and using inductively coupled plasma atomic emission spectroscopy (ICP-AES, Agilent 5100 Dual View) of the aqueous phase. Samples of the aqueous phase were collected before the LLE and after 5, 15, 30, and 45 minutes of LLE. 0.35 mL of each sample was digested in 5 mL with 10% (v/v) H₂SO₄ for 6 hours at 160° C. 2.7 mL of the digested sample was diluted up to 10 mL with Milli-Q water to reach a total acid concentration of 5% (v/v). Calibration standards (Inorganic Ventures) were prepared to match the sample matrix (Sc) with concentrations of 22.2, 18, 15, 10, and 5 ppm Ti and Sc and run prior to every set of samples. Samples were analyzed in radial view at a viewing height of 8 mm. The extraction to the organic phase was calculated from the concentration of Ti and Sc in the aqueous phase before and after the LLE by E=((c_{before} LLE−c_{after} LLE)/c_{before} LLE)−100%.

Batch LLE and Separation

For the non-radioactive work, 0.5 mL 0.01 M TiCl₄ and 0.01 M ScCl₃ in 37% HCl was mixed with extractant(s) and shaken for 2 min in a centrifuge tube. The phases were allowed to separate by gravity. The concentration of Ti and Sc in the aqueous phase before and after the LLE was measured by ICP-AES.

For the batch LLE of ⁴⁵Ti, a centrifuge tube was charged with 2 mL of the solution of ⁴⁵Ti (10-50 MBq) in 37% HCl and 2 mL of the organic phase. The mixture was shaken vigorously, spun for 15 minutes, and centrifuged at 4000 rpm to separate the phases.

An Eppendorf 5702 centrifuge was used to assist in phase separation. For batch experiments, the phase mixing was performed using a IKA ROCKER 3D digital shaker.

Continuous Membrane-Based LLE and Separation

The continuous liquid-liquid extraction and phase separation in flow was performed using a membrane-based separator with a PFA diaphragm for integrated pressure control. A flow schematic of the apparatus is depicted in FIG. 1. The two phases passed through PFA tubing ( 1/16″ (1.5875 mm) OD, 0.03″ (0.762 mm) ID) and were mixed in a PEEK tee, followed by two 10 element PTFE static mixers (3.4 cm total length) and various lengths of PFA mixing tubing, which were used to control the residence time of the LLE. After the static mixers, steady liquid-liquid segmented flow was developed and passed through the LLE mixing loop and finally into the membrane separator, where the organic phase permeated the membrane while the aqueous phase was retained. Different diaphragm thicknesses, membranes and flow rates were tuned to achieve complete separation of the aqueous and organic phases, as described in detail below.

### Example 1—Investigation of Extractants for Batch Liquid-Liquid Extraction of Ti Ions from a Solution Also Containing Sc Ions

The preliminary screening experiments were performed in batch using gravity separation. It had been reported⁶⁴ that ⁴⁵Ti can be extracted from aqueous HCl into 1-octanol, presumably as [⁴⁵Ti]Ti n-octyloxide 1, Structure 1.

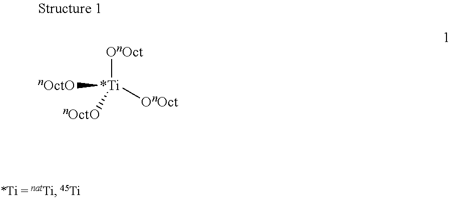

TABLE 1
Liquid-liquid batch extraction of ⁴⁵Ti from cyclotron-irradiated
Sc foil digested in 37% HCl, except entry 1a.

| Entry | Extraction system (organic phase) | D | EE (%) |
|---|---|---|---|
| 1 | 1-octanol, neat^{a} | 0.04 | 4 |
| 2 | 1-octanol, neat | 0.88 | 47 |
| 3 | 1,2-Decanediol, 0.1M in 1-octanol | 1.2 | 54 |
| 4 | 2,3-naphthalene diol, 0.1M in 1-octanol | 1.1 | 52 |
| 5 | C₁₀F₂₁CH₂CH(OH)CH₂OH^{b} | <0.001 | <0.1 |
| 6 | Guaiacol, neat | 3 | 75 |

D is the distribution coefficient,
D = [⁴⁵Ti(org)]/[⁴⁵Ti(aq)] and EE is the extraction efficiency,
EE = 100% * [⁴⁵Ti(org)]/[⁴⁵Ti(total)] as measured by a radiation detector.
^{a}20% HCl;
^{b}trifluorotoluene/hexafluoropropanol (1/1, (v/v))

We observed little extraction when a solution of ⁴⁵Ti in 20% HCl was used (Table 1, entry 1). Using 37% HCl (12 M) significantly improved the extraction. 1,2-Decanediol used as co-extractant as a 0.1 M solution in 1-octanol gave only a slight improvement over neat 1-octanol. 2,3-naphthalene diol, reported, in the context of the extractive spectrophotometric determination of Ti content in rocks, to extract Ti at pH=4,⁶⁵ showed similarly modest performance. (Table 1, entries 3 and 4). During these batch extractions, we noticed a significant increase in the volume of the 1-octanol phase suggestive that conc. HCl was migrating into the organic phase. In an attempt to improve the phase separation we turned to perfluorinated extractants.⁶⁶ Disappointingly, the fluorous analog of 1-octanol, CF₃(CF₂)₅CH₂CH₂OH, formed an emulsion. A 0.05 M solution of C₁₀F₂₁CH₂CH(OH)CH₂OH in trifluorotoluene/hexafluoropropanol (1/1, (v/v)) failed to extract any ⁴⁵Ti. (Table 1, entry 5). Previously, it was reported, in the context of investigating catalytic complexes of Ti, that guaiacol (o-methoxyphenol) and maltol were able easily to form moisture-sensitive but isolable complexes with titanium tetrachloride.⁶⁷ Gratifyingly, using neat guaiacol as an extractant, we were able to extract 75% of activity into the organic phase, presumably as Structure 2.

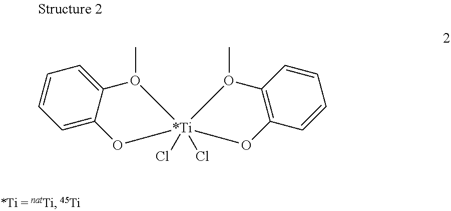

### Example 2—Investigation of Extractants and Conditions for Liquid-Liquid Extraction and Phase Separation in Flow of Ti Ions from a Solution Also Containing Sc Ions

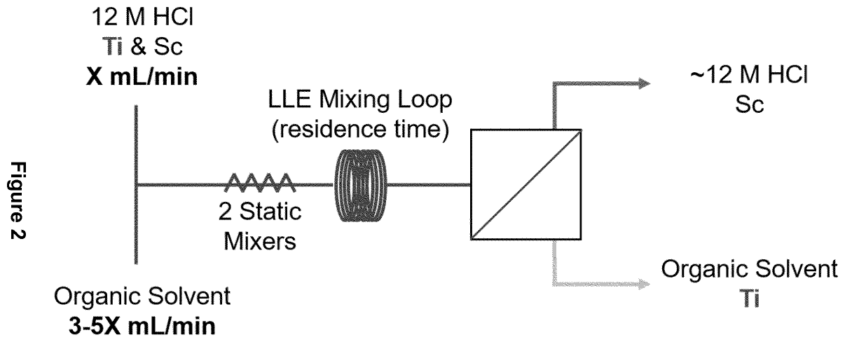

The LLE and phase separation in flow were performed using a membrane-based separator with a PFA diaphragm for integrated pressure control (FIG. 2).

A flow schematic of the experimental setup is analogous to what has been depicted in FIG. 2. In short, the two phases passed through PFA tubing ( 1/16″ (1.5875 mm) OD, 0.03″ (0.762 mm) ID) and were mixed in a PEEK tee, followed by two 10 element PTFE static mixers (3.4 cm total length) and various lengths of PFA mixing tubing, which were used to control the residence time of the LLE. After the static mixers, steady liquid-liquid segmented flow was developed and passed through the mixing loop and finally into the membrane separator, where the organic phase permeated the membrane while the aqueous phase was retained.

To find the optimum extraction conditions, the diaphragm thickness, membrane pore size, organic phase composition, flow rate ratios, and residence times were varied. Once the optimal materials and conditions were determined, a study was conducted to determine the shortest residence time, thereby minimizing the dead volume and overall processing time, while still maintaining high extraction. The residence time was varied by varying the length of the mixing tubing after the static mixers while maintaining a constant flow rate. All systems were operated for 60 min each and samples were collected every 15 min.

Investigation of Membrane Pore Size, Diaphragm Thickness and Organic Phase Composition

Due to the harsh nature of both the aqueous and organic solvents used for this extraction, both the membrane and diaphragm had to be extremely stable. Therefore, PTFE membranes were used for all experiments described herein.

Complete separation requires both the diaphragm thickness and membrane pore size be chosen so that P_{dia} lies between the P_{cap} and Pₚₑᵣₘ pressures. In general, low interfacial tension mixtures often require smaller pore size membranes and thinner diaphragms. In these experiments, polytetrafluoroethylene (PTFE) membranes were tested using the following pore sizes: 0.1, 0.2, and 0.5 μm. Three different diaphragm film thicknesses were also tested: 0.001″, 0.002″, and 0.005″ (0.025 mm, 0.051 mm, and 0.127 mm).

Since guaiacol had shown the most selective and highest extraction efficiency for Ti over Sc in batch, it was chosen as a candidate for translation into flow, with 1-octanol also being investigated.

TABLE 2

| | | |
|---|---|---|
| Membrane | Diaphragm | |
| Pore Size | Thickness | |
| [μm] | [in (mm)] | Performance |

37% HCl and 1-octanol

| | | |
|---|---|---|
| 0.5 | 0.005 (0.127) | Breakthrough |
| | 0.002 (0.051) | Breakthrough |
| | 0.001 (0.025) | Breakthrough |
| 0.2 | 0.001 (0.025) | Breakthrough |
| 0.1 | 0.001 (0.025) | Retention/Breakthrough |

Separation performance for various membrane pore sizes and diaphragm thicknesses for 37% HCl mixed with 1-octanol

It can be seen from Table 2 that no satisfactory conditions could be found for the use of 1-octanol as extractant. As the extraction efficiency was only around 50%, it was decided not to investigate this extractant further.

A solution of 0.01 M TiCl₄ and 0.01 M ScCl₃ in 37% HCl was extracted into guaiacol using the membrane separator.

Occasional retention and/or breakthrough of the aqueous into the organic phase was observed with a 0.2 μm PTFE/PP membrane, 0.002″ (0.051 mm) diaphragm thickness, and 0.2 mL/min total flow rate. The situation was remedied by adding various amounts of anisole as an interfacial tension modifier, which is structurally similar to guaiacol but acted to increase the interfacial tension, as shown in Table 3. Although the guaiacol/anisole mixture performed much better than 1-octanol, its extraction efficiency was not high enough. Therefore, the ratio of guaiacol to anisole was varied as well, as summarized in Table 5.

TABLE 3

| | | |
|---|---|---|
| Membrane | Diaphragm | |
| Pore Size | Thickness | |
| [μm] | [in (mm)] | Performance |

37% HCl and 1:1 Guiacol/Anisole

| | | |
|---|---|---|
| 0.5 | 0.005 (0.127) | Breakthrough |
| | 0.002 (0.051) | Breakthrough |
| | 0.001 (0.025) | Retention |
| 0.2 | 0.002 (0.051) | Complete separation |

Separation performance for various membrane pore sizes and diaphragm thicknesses for 37% HCl mixed with 1:1 guaiacol/anisole.

Investigation of Flow Rate Ratios and Organic Phase Compositions Since a 0.2 μm membrane and a 0.002″ (0.051 mm) diaphragm was the only combination that led to complete phase separation, it was used for all of the optimization experiments.

The organic mixtures of guaiacol and anisole were used to extract Ti, (0.01 M) from 37% HCl at a total flow rate of 0.20 mL/min and aqueous to organic ratios of 1/5, 1/3, 1/1, 3/1, and 5/1 (v/v). Corresponding flow rates are shown in Table 4.

TABLE 4

| Aq/Org | Aq. Flow | Org. Flow |
|---|---|---|
| Flow Ratio | Rate Ratio | Rate Ratio |
| [—] | [mL/min] | [mL/min] |
| 1:5 | 0.03 | 0.17 |
| 1:3 | 0.05 | 0.15 |
| 1:1 | 0.10 | 0.10 |
| 3:1 | 0.15 | 0.05 |
| 5:1 | 0.17 | 0.03 |

Aqueous to organic flow rate ratios with corresponding volumetric flow rates (total flow rate = 0.20 mL/min).

The composition of the organic phase needed to both selectively extract only Ti and have a high enough interfacial tension with the HCl phase that complete separation could be achieved. It was determined that extraction was directly correlated with guaiacol concentration, that is a higher guaiacol concentration led to higher extraction up to a maximum Ti extraction of 90% with 90% guaiacol. Guaiacol concentrations above 90% led to incomplete phase separation. A summary of the phase separation performance using various organic phase compositions is shown in Table 5.

In addition to the composition of the organic phase, the relative ratios of aqueous to organic flow rates were also varied. When comparing relative flow rate ratios of 1/1, 1/3, and 1/5 (v/v) (aq. to org.) it was determined that 1/1 gave the lowest extraction. A ratio of 1/3 gave a higher extraction, but 1/5 did not yield a further increase in performance. All ratios where the aqueous flow rate was higher led to lower extraction efficiency. Therefore, a flow rate ratio of 1/3 was chosen as to avoid using excess solvent (Table 5).

TABLE 5

| | | | |
|---|---|---|---|
| Org. Phase | Aq. | Org. | |
| Guaiacol/Anisole | Flow Rate | Flow Rate | Performance |
| (v/v) | [mL/min] | [mL/min] | [—] |

Aqueous Phase: 37% HCl (12M)
0.2 μm PTFE membrane and 0.002” (0.051 mm) Diaphragm thickness

| | | | |
|---|---|---|---|
| 10/90 | 0.10 | 0.10 | Complete Sep. |
| 75/25 | 0.05 | 0.15 | Complete Sep. |
| | 0.10 | 0.10 | |
| | 0.10 | 0.30 | |
| | 0.25 | 0.75 | |
| 90/10 | 0.03 | 0.17 | Complete Sep. |
| | 0.05 | 0.15 | |
| | 0.25 | 0.75 | |
| 95/05 | 0.10 | 0.10 | Retention/Breakthrough |

Phase separation performance for different guaiacol to anisole ratios in the organic phase using different aqueous to organic flow rate ratios.

Investigation of Scalability and Stability of the Extraction

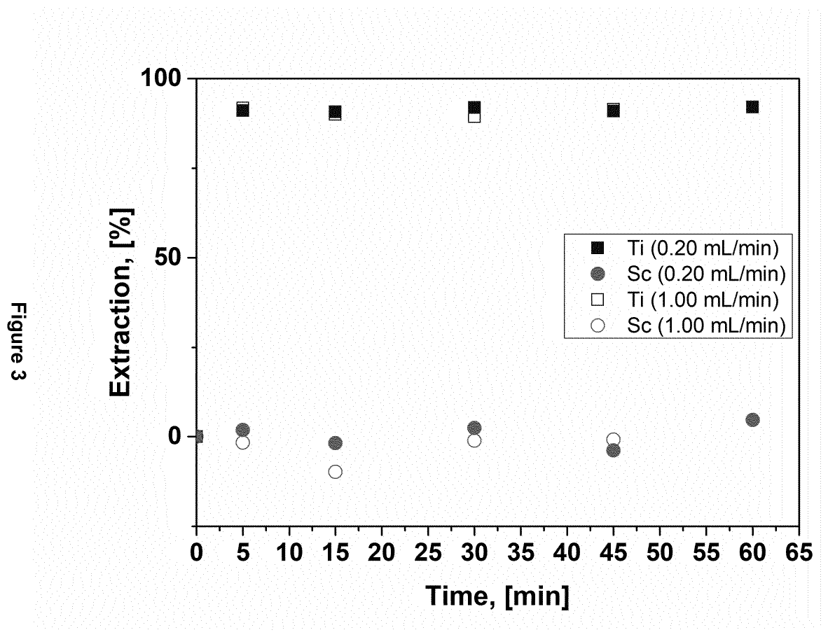

In order to determine the scalability and stability of the extraction, the total flow rates were scaled five-fold to a total flow rate of 1.00 mL/min, while maintaining the same flow rate ratios and residence times. The extraction performance was identical to the original scale, and the maximum extraction of 90% was still achieved at 90% guaiacol and a flow rate ratio of 1/3 (FIG. 3).

Investigation of Residence Time

After an optimal system was developed, the residence time of mixing was varied to minimize the dead volume and decrease the total amount of time spent in the system. This was achieved by increasing or decreasing the length of the PFA tubing used for mixing. The following lengths were tested with their corresponding residence times at 0.20 mL/min: 10 cm (13.7 s), 25 cm (34.2 s), 54 cm (73.9 s), 108 cm (147.8 s), 216 cm (295.6 s).

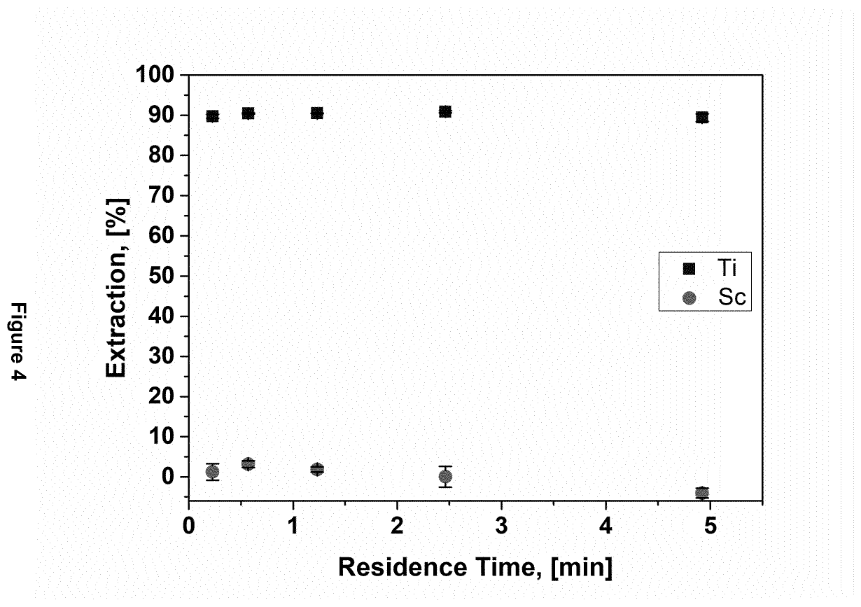

The total production time is a critical parameter in this system since the radioactive Ti is continuously undergoing decay back to Sc (t½=3 hours). Therefore, the shortest possible residence time is desirable. Residence times of the mixing tubing were varied from 13.7 s up to −5 min. The extraction efficiency was the same for all residence times. Therefore the shortest residence time was the most optimal, and resulted in a total system residence time of less than 1 min (FIG. 4).

Conclusion—Optimal Conditions

Overall, an organic phase consisting of 90% guaiacol and 10% anisole, total flow rates of 0.20 or 1.00 mL/min, an aqueous to organic flow rate ratio of 1/3, and a residence time of 13.7 s led to highest and most efficient extraction resulting in 90.3±1.1% extraction of Ti and 0% extraction of Sc.

### Example 3—Optimisation of Conditions for Liquid-Liquid Extraction and Phase Separation in Flow of ⁴⁵Ti Ions from a Solution Also Containing Sc Ions

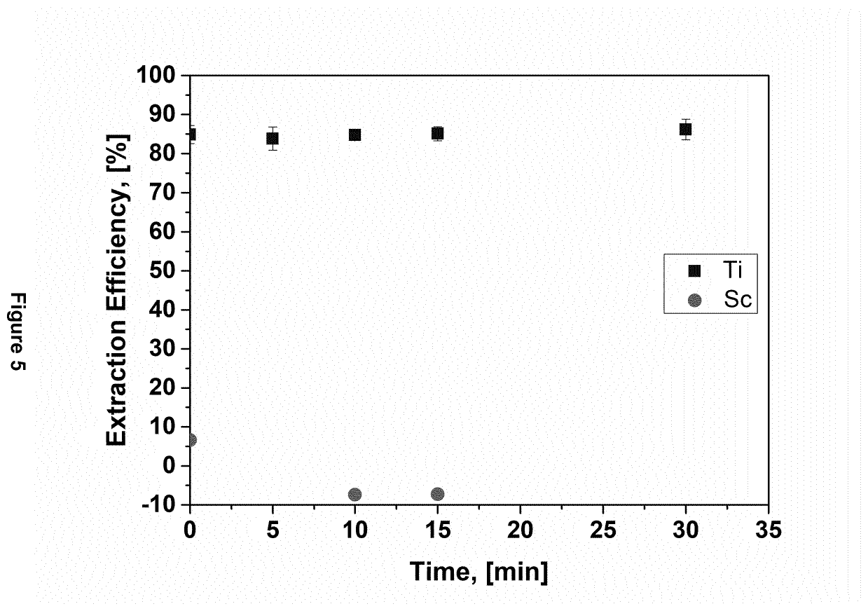

With the extraction conditions optimized for ^{nat}Ti, we turned to the radioactive isotopomer, ⁴⁵Ti. While the concentration of Sc was comparable in both non-radioactive and radioactive cases (0.01 M vs. 0.03-0.13 M correspondingly) the concentration of the radiometal in the Sc-containing matrix solution was lower than that of its natural abundance isotopomer by 10 orders of magnitude, ranging from 1 to 10 picomoles. At these concentrations, even trace levels of impurities or water could potentially lead to side-reaction or hydrolysis and, as a consequence, change the extraction efficiencies of ⁴⁵Ti. To our delight, however, the LLE of ⁴⁵Ti in flow using a guaiacol-anisole 9/1 (v/v) mixture and a flow rate ratio of 1/3 (aq. to org.), with a residence time of 13.7 s showed that the extraction efficiency of ⁴⁵Ti was consistent with that of ^{nat}Ti (84.8±2.4% and 90.3±1.1% correspondingly), (FIG. 5). The ICP-AES analysis of the aqueous phase before and after the LLE confirmed that no Sc was extracted into the organic phase.

### Example 4—Synthesis of ⁴⁵Ti-Containing Radiotracer

Finally, to examine if the extracted solution of ⁴⁵Ti can be directly used for radiolabelling, we attempted a synthesis of [⁴⁵Ti](salan)Ti(dipic) 3, a Ti-antineoplastic, previously used for animal ⁴⁵Ti-PET and ex vivo radiotracing (Scheme 1).¹⁸

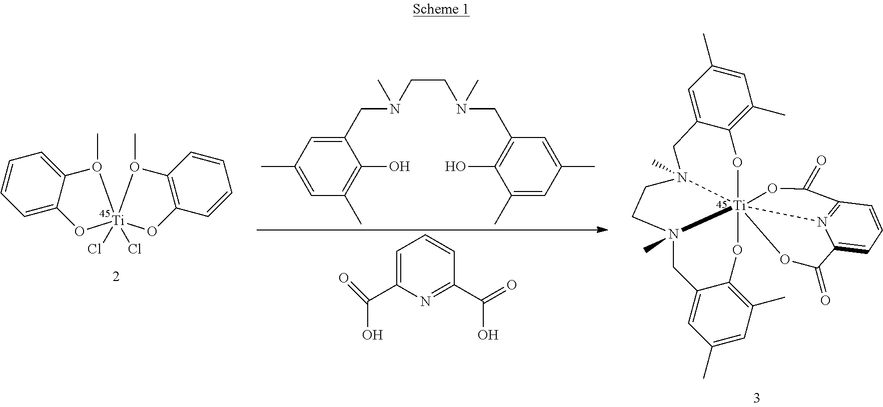

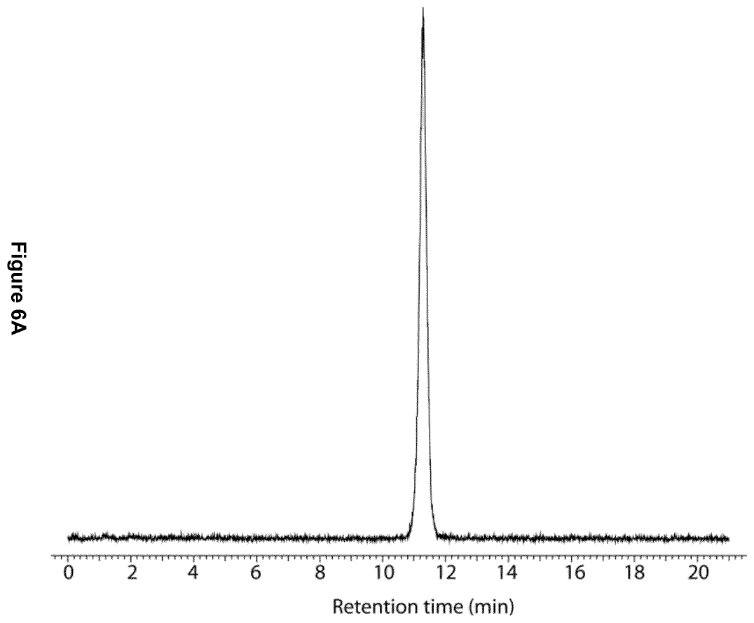

To that end, the organic phase after the continuous LLE of ⁴⁵Ti was collected and reacted with an equimolar solution of salan and 2,6-pyridine dicarboxylic acid (dipic) in pyridine at 60° C. An essentially complete (98.7%) conversion to the desired product 3 was observed within 15 min as evidenced by radio-TLC (red peak for the product 3 and only traces of unreacted 2, green peak), proving the high quality and reactivity of extracted ⁴⁵Ti (FIG. 6B) The HPLC/radio-HPLC further confirmed the identity of the product 3 matching its retention time to that of the independently synthesized non-radioactive [^{nat}Ti](salan)Ti(dipic) determined by HPLC equipped with a UV-detector (FIG. 6A).

Separation of Zr and Y

Materials

Guaiacol (99%, natural), anisole (99%, ReagentPlus), hydrochloric acid (37%, ACS reagent), sulfuric acid (95.0-98.0%), and trioctylphosphine oxide (TOPO, ≥98.5%) were purchased from Sigma Aldrich. High purity hydrochloric acid (37%, “Ultrapur”) was purchased from Merck. All purchased chemicals were used without further purification. Yttrium foil (99.9%) was purchased from Alfa Aesar. ZrCl₄ and YCl₃ were purchased from Sigma Aldrich.

Pall PTFE membranes were used for all experiments (47 mm diameter, 0.1/0.2/0.5 μm pore size, polypropylene support). Perfluoroalkoxy alkane (PFA) diaphragms (0.001″/0.002″/0.005″ (0.0254/0.0508/0.1270 mm)) were purchased from McMaster Carr. All PFA tubing ( 1/16″ (1.5875 mm) OD, 0.03″ (0.762 mm) ID) was purchased from Idex Health and Science. PTFE static mixers were purchased from Stamixco. Two syringe pumps (KDS 100 Legacy Syringe Pump) and a dose calibrator (CRC-55tR, CII Capintec, Inc.) were used for the experiments.

Radionuclide Production and Separation

For all experiments, the cyclotron target material (yttrium) was used at its natural abundance level.

⁸⁹Zr was produced by proton bombardment of yttrium foils on a PETTrace PT800 cyclotron. The 640 μm thick, 5 mm×5 mm foils were cut and sandwiched between a silver disc and a 500 μm Al degrader and placed in the target holder, providing direct water cooling on the rear face of the silver. Based on SRIM calculations the Al foil degrades the incident proton energy from the nominal 16.5 to approx. 13.1 MeV, bringing the energy below the threshold for co-production (<100 μb) of both ⁸⁸Y and ⁸⁸Zr. The irradiated foil was digested in 30-37% HCl. The mixture was filtered and centrifuged if necessary. If needed, the solution was diluted with water to make the final dilution ca. 6 M in HCl. These dilutions were used as the aqueous phase for the LLEF.

Instrumentation and Methods

For ⁸⁹Zr work, the extent of extraction (extraction %) was determined from the radioactivity measurements and using inductively coupled plasma atomic emission spectroscopy (ICP-AES, Agilent 5100 Dual View) of the aqueous phase. Samples of the aqueous phase were collected before the LLE and after 5, 15, 30, and 45 minutes of LLE. 0.35 mL of each sample was digested in 5 mL with 10% (v/v) H₂SO₄ for 6 hours at 160° C. 2.7 mL of the digested sample was diluted up to 10 mL with Milli-Q water to reach a total acid concentration of 5% (v/v).

An Eppendorf 5702 centrifuge was used to assist in phase separation. The membrane separator module was similar to those manufactured by Zaiput Flow Technologies. The solutions for the continuous membrane-based separation were pumped using either the KDS 100 Legacy Syringe (radioactive experiments) or the Harvard Apparatus PHD 2000 Programmable and Infusion syringe pumps (non-radioactive experiments). For batch experiments, the phase mixing was performed using an IKA ROCKER 3D digital shaker.

The continuous liquid-liquid extraction in flow (LLEF) was performed using the apparatus depicted in FIG. 1. The aqueous and the organic phases were combined through a tee and mixed with two static mixers and mixing tubing. The aqueous phase was retained by the membrane, while the organic phase permeated through the membrane. Under the conditions of direct extraction, the radionuclide (⁸⁹Zr) was selectively extracted over yttrium into the organic phase.

### Example 5—Investigation of Extractants and Conditions for Liquid-Liquid Extraction and Phase Separation in Flow of Zr Ions from a Solution Also Containing Y Ions

Earlier reports indicated that zirconium can be extracted from its acidic solutions into an organic phase containing trioctylphosphine oxide (TOPO).³⁵ However, the preliminary batch experiments containing the equimolar solutions of 0.01 M ZrCl₄ and YCl₃ in 37% HCl produced a 3-phase mixture.

We began by testing whether the phase separation in this extraction system can be improved under the LLEF conditions.

Starting with the conditions optimized for Ti/Sc extraction (0.2 μm membrane, 0.002″ (0.051 mm) diaphragm, and 0.05/0.15 mL/min aq/org flow rate) we found that extensive breakthrough of the third phase occurred at both 0.2 μm and 0.1 μm membrane pore size (Table 6, entry 1). Lowering the concentrations of ZrCl₄ and YCl₃ to 0.001 M and then to 0.0005 M did not result in any improvement in the phase separation either (Table 6, entries 3-4). Only by lowering the concentration of HCl from 12 M to 6 M can a complete phase separation be achieved (Table 6, entry 4).

Unable to use the literature conditions for LLEF of Zr, we turned to 9/1, (v/v) guaiacol/anisole mixture, which performed extremely well for the phase separation and LLEF of Ti. Complete phase separation occurred in 37% HCl at both 0.001 M and 0.01 M ZrCl₄ and YCl₃ in a wide range of flow rates (Table 6, entries 5-6).

TABLE 6

| | | | Flow rate | Flow rate | |
|---|---|---|---|---|---|
| | | Aqueous | (aqueous) | (organic) | Separation |
| Entry | Organic phase | phase | (mL/min) | (mL/min) | performance |
| 1 | 0.1M TOPO | 37% HCl | 0.05 | 0.15 | Breakthrough |
| | in hexane | | | | (third phase) |
| 2 | 0.1M TOPO | 0.001M ZrCl₄ and | 0.05 | 0.15 | Breakthrough |
| | in hexane | YCl₃ in 37% HCl | | | (third phase) |
| 3 | 0.1M TOPO | 0.0005M ZrCl₄ and | 0.05 | 0.15 | Breakthrough |
| | in hexane | YCl₃ in 37% HCl | | | (third phase) |
| 4 | 0.1M TOPO | 0.001M ZrCl₄ and | 0.05 | 0.15 | Complete |
| | in hexane | YCl₃ in 6M HCl | | | phase |
| | | | | | separation |
| 5 | 90% guaiacol | 0.001M ZrCl₄ and | 0.033 | 0.167 | Complete |
| | 10% anisole | YCl₃ in 37% HCl | | | phase |
| | | | | | separation |
| 6 | 90% guaiacol | 0.01M ZrCl₄ and | 0.05 | 0.15 | Complete |
| | 10% anisole | YCl₃ in 37% HCl | 0.25 | 0.75 | phase |
| | | | 0.033 | 0.167 | separation |

Phase separation performance for different extractant/interfacial tension modifier mixtures in the organic phase, different starting concentrations in the aqueous phase, and using different aqueous to organic flow rate ratios.

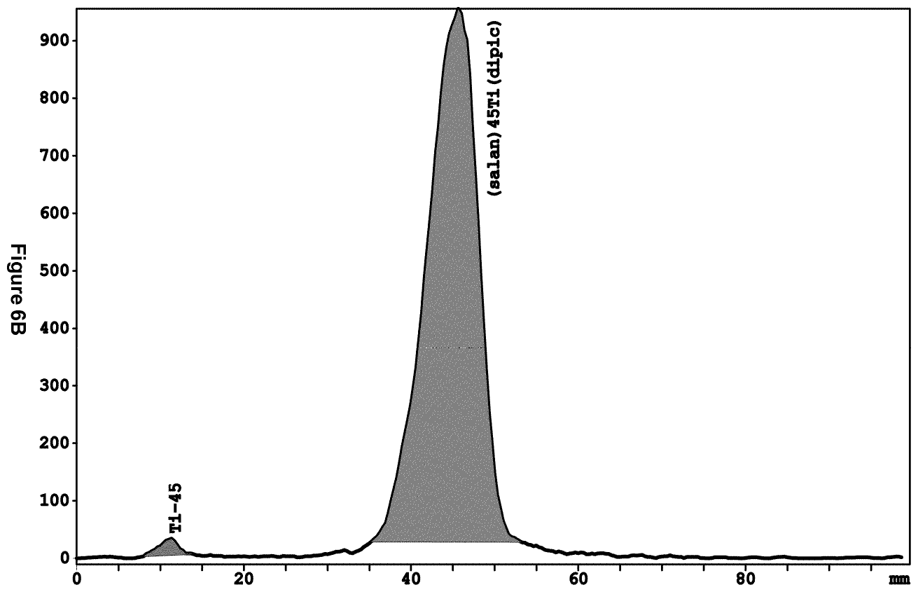

The extraction efficiencies of Zr from 0.01 M ZrCl₄ solution in 37% HCl, also containing 0.01 M YCl₃ were investigated at different flow rates using the guaiacol/anisole, 9/1 (v/v) mixtures (FIG. 7). Whereas no significant difference was found between low (0.05/0.15 mL min aq/org) and high (0.25/0.75 mL min aq/org) flow rates at 1/3 aq/org ratios, an increase in Zr extraction up to 72% was observed at the 1/5 aq/org ratio. The extraction of yttrium was below the limit of detection.

### Example 6—Investigation of Extractants and Conditions for Liquid-Liquid Extraction and Phase Separation in Flow of ⁸⁹Zr Ions from a Solution Also Containing Y Ions

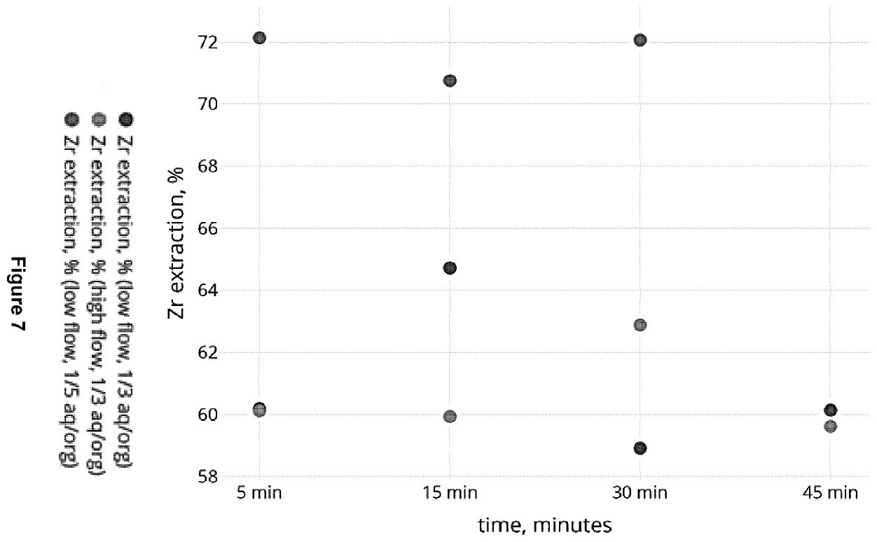

The extraction of ⁸⁹Zr from its solution in 37% HCl, also containing 0.01 M YCl₃ was explored at low flow rates (0.033/0.166 mL/min, aq/org) using the guaiacol/anisole, 9/1 (v/v) mixtures. (FIG. 8). The extraction efficiency as the average of three runs was similar to that obtained at the natural abundance level experiments.

Separation of Gallium and Zinc

Materials

Anisole (99%, ReagentPlus), hydrochloric acid (37%, ACS reagent), zinc chloride (≥98%), sulfuric acid (95.0-98.0%), dibutyl ether, butyl methyl ether, tetrahydropyran, hexyl methyl ether, α,α,α-trifluorotoluene, and toluene, were purchased from Sigma Aldrich. Diethyl ether, diisopropyl ether (≥99%), and high purity hydrochloric acid (37%, “Ultrapur”) were purchased from Merck. Heptane (99.7%) and 1,2-dichloroethane were purchased from VWR Chemicals. All purchased chemicals were used without further purification. Zinc foil (99.9%) was purchased from Alfa Aesar.

Pall PTFE membranes were used for all experiments (47 mm diameter, 0.1/0.2/0.5 μm pore size, polypropylene support). Perfluoroalkoxy alkane (PFA) diaphragms (0.001″/0.002″/0.005″ (0.0254/0.0508/0.1270 mm)) were purchased from McMaster Carr. All PFA tubing ( 1/16″ (1.5875 mm) OD, 0.03″ (0.762 mm) ID) was purchased from Idex Health and Science. PTFE static mixers were purchased from Stamixco. The 15 mL plastic centrifuge tubes with screw caps were purchased from VWR.

Combined Radiogallium (⁶⁶Ga, ⁶⁷Ga, ⁶⁸Ga) and ⁶⁵Zn Production and Purification

For all experiments, the cyclotron target material (zinc) was used at its natural abundance level.

Production: These radionuclides were produced simultaneously, by proton bombardment of stacked Zn and Cu foils. The incident 16.5 MeV proton beam would first encounter a 250 μm thick, 831 mg Zn foil before entering a 500 μm thick, 327 mg Cu foil. Incident energy on the Cu foil was calculated to appx. 12.8 MeV, making the 500 μm foil a thick target (range in Cu only 370 μm). The foils were irradiated for 160 minutes at 10 μA resulting in an integrated current of 26.2 μAh. The irradiated Zn foil, containing gallium radioisotopes was dissolved in a small amount of 3 M or 6 M hydrochloric acid and then added to either the 7 molal (m) or 1 M stock solution of ZnCl₂ also prepared in 3 M or 6 M hydrochloric acid.

⁶⁵Zn Purification: The irradiated Cu foil (327 mg, containing 5.6 MBq of ⁶⁵Zn) was dissolved in 1.7 mL of concentrated HNO₃ at 60° C. The deep blue solution was evaporated to dryness at 150° C. using vigorous Ar flow. The blue solid was re-dissolved in 2.5 mL 1 M HCl, and loaded onto TK200 resin (3 g). The resin was first eluted with 1 M HCl, which removed all the copper (a total of 14 mL), and then with water, which eluted the zinc (a total of 25 mL). The fractions containing the highest amount of ⁶⁵Zn were collected, the solution was evaporated to dryness, and added to either the 7 molal (m) or 1 M stock solution of ZnCl₂ prepared as described above. The resulting solution, containing 100-300 kBq of ⁶⁵Zn and radiogallium (present mostly as ⁶⁷Ga) and simulating a cyclotron-irradiated liquid target mixture was used as the aqueous phase for the LLE.

Instrumentation and Methods

Gallium and zinc were quantified by measuring radioactivities from ⁶⁷Ga, ⁶⁸Ga, and ⁶⁵Zn radioisotopes using the CRC-55tR, CII (Capintec, Inc) dose calibrator and Princeton Gammatech LGC 5 and Ortec GMX 35195-P gamma spectrometers.

An Eppendorf 5702 centrifuge was used to assist in phase separation. The membrane separator module was similar to those manufactured by Zaiput Flow Technologies. The solutions for the continuous membrane-based separation were pumped using either the KDS 100 Legacy Syringe (radioactive experiments) or the Harvard Apparatus PHD 2000 Programmable and Infusion syringe pumps (non-radioactive experiments). For batch experiments, the phase mixing was performed using an IKA ROCKER 3D digital shaker.

For the phase separation studies, a centrifuge tube was charged with 1.3 mL of the 7 m ZnCl₂-3 M HCl, or 7 m ZnCl₂-6 M HCl solution and various amounts of organic phase were added. The mixture was shaken for 30 minutes and centrifuged at 4000 rpm to separate the phases.

For the batch LLE of gallium, a centrifuge tube was charged with 1.3 mL of the 7 m ZnCl₂-3 M HCl, or 7 m ZnCl₂-6 M HCl solution, also containing ⁶⁷Ga, ⁶⁸Ga, and ⁶⁵Zn radioisotopes, and various amounts of organic phase were added. The mixture was shaken for 30 minutes and centrifuged at 4000 rpm to separate the phases.

The continuous liquid-liquid extraction and phase separation in flow was performed using a membrane-based separator with a PFA diaphragm for integrated pressure control. A flow schematic of the apparatus is depicted in FIG. 1. The aqueous and the organic phases were combined through a tee and mixed with two static mixers and mixing tubing. The aqueous phase was retained by the membrane, while the organic phase permeated through the membrane. Under the conditions of direct extraction, the radionuclide (⁶⁸Ga) was selectively extracted over zinc into the organic phase. Under the conditions of reverse extraction, also known as back-extraction or stripping, the radionuclide ⁶⁸Ga was extracted together with zinc into the aqueous (0.1 M HCl) phase. To provide for additional purification of gallium the direct LLEF of residual zinc from the organic phase into 8 M HCl was also performed. This process is also known as scrubbing.

### Example 7—Phase Separation Studies Using Several Dialkyl Ethers and Hydrochloric Acid, Also Containing Concentrated Zinc Chloride

The earlier work established that dialkyl ethers, and in particular diethyl ether, efficiently and selectively extracted gallium from 5-6 M hydrochloric acid solutions in batch.⁶⁸⁻⁷¹ Since dialkyl ethers are generally non-toxic, readily available low boiling point liquids, we decided to evaluate this class of compounds for further development in LLE and membrane-based separation of gallium from zinc. Tetrahydropyran (THP) diethyl (Et₂O), diisopropyl (ⁱPr₂O), dibutyl (Bu₂O), butyl methyl (BuOMe), and hexyl methyl (HexOMe) ethers were chosen as the extractants. The preliminary experiments showed that the presence of concentrated (7 m) ZnCl₂ dramatically influenced the phase equilibrium. A single phase was observed by mixing equal volumes of diethyl ether, and a concentrated solution of ZnCl₂ prepared in 6 M HCl. Lowering the concentration of HCl to 5, and then to 4 M still produced a single phase. At 3.5 M HCl two phases finally separated but extensive migration of aqueous into the organic phase was observed (Table 7, entry 8, Et₂O/aq=6.22, (v/v)). Lowering the concentration of HCl further led to a gradual decrease in the ratio Et₂O/aq, (v/v), ie a decrease in the migration of aqueous into the organic phase (Table 7, entries 2-4). Unexpectedly, this trend was opposite to what one observed when ZnCl₂ was not present.⁶⁹ The extraction efficiency of Ga and Ga/Zn selectivity were also disappointing.

TABLE 7

| | | | Extraction in Et₂O (%) | |
|---|---|---|---|---|
| Entry | HCl (M) | Et₂O/aq, (v/v) | ⁶⁸Zn | ⁶⁸Ga |
| 1 | 0 | 1.02 | 15.29 | 8.23 |
| 2 | 0.5 | 1.21 | 61.51 | 22.09 |
| 3 | 1 | 1.51 | 66.47 | 28.9 |
| 4 | 1.5 | 1.74 | | |
| 5 | 2 | 2.02 | 73.19 | 47.37 |
| 6 | 2.5 | 2.58 | | |
| 7 | 3 | 3.82 | 80.24 | 64.91 |
| 8 | 3.5 | 6.22 | | |

Batch extraction of zinc and gallium into diethyl ether from a solution of ZnCl₂ prepared by dissolving 1 g of salt in 1 mL of aqueous HCl of a given strength.

### Example 8—the Effect of Adding an Interfacial Tension Modifier on Phase Separation Using Several Dialkyl Ethers and Hydrochloric Acid, Also Containing 7 m Zinc Chloride

The interfacial migration is a critical parameter which had to be minimized to prevent the contamination of aqueous phase with the organic phase, which would make the process incompatible with the implementation of continuous LLEF due to stringent organic-free requirements for the ZnCl₂-based aqueous cyclotron solution targets. The significant interfacial migration would also lead to low interfacial tension, which might cause a phase breakthrough during membrane separation. Our strategy was to find a suitable interfacial tension modifier which provided for reliable phase separation with no or little interfacial migration while keeping good Ga extraction efficiency and high Ga/Zn selectivity. Given its low capacity to dissolve water⁷², toluene was initially chosen as an interfacial tension modifier for screening the phase separation in the series R₁OR₂/ZnCl₂—HCl.

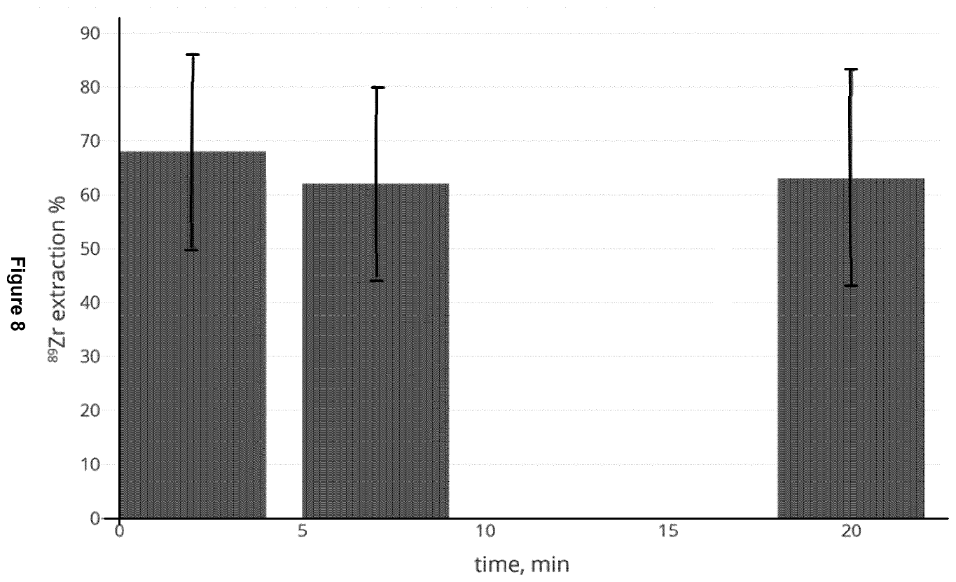

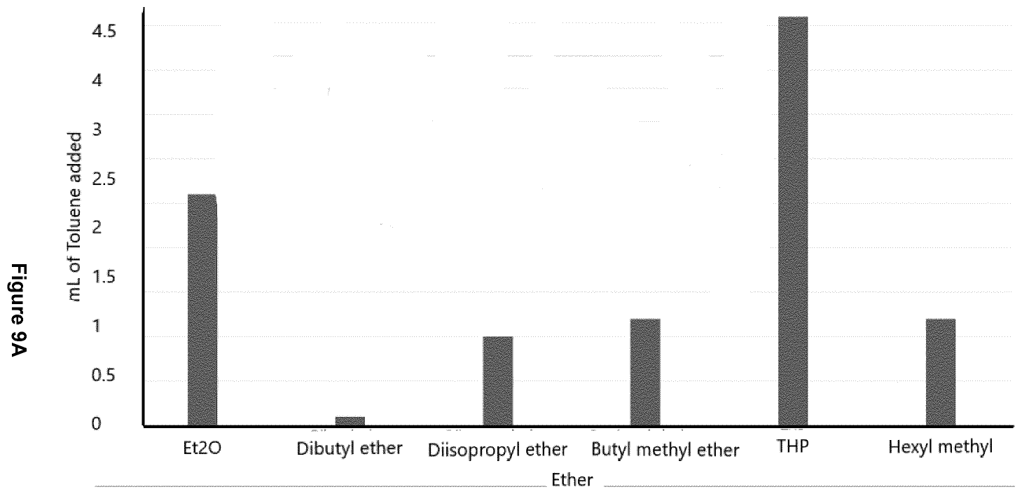

FIG. 9 shows the amount of toluene which had to be added to a 1/1 (v/v) mixture of R₁OR₂ and ZnCl₂-6M HCl to achieve complete phase separation. THP had the highest affinity for the aqueous phase, and Bu₂O required no or little interfacial tension modifier. Using THP as the worst phase separation extractant, we screened a series of interfacial tension modifiers chosen from five major classes of organic solvents and represented by toluene, anisole, dichloroethane, trifluorotoluene, and heptane. Quite counter-intuitively, it took the largest amount of heptane to achieve the desired phase separation in 6 M HCl (FIG. 9B, A, lilac). As an interfacial tension modifier, TFT was the best overall, being uniquely insensitive to HCl concentration (FIG. 9B, red).

### Example 9—the Batch Extraction of Ga and Zn Using Several Dialkyl Ethers, TFT, and Hydrochloric Acid, Also Containing 7 m Zinc Chloride

Having established a preferred interfacial tension modifier for phase separation, we investigated its performance for Ga/Zn extraction selectivity in the series of extractants R₁OR₂/ZnCl₂—HCl at 6 M and 3 M HCl. Table 8 shows that TFT in combination with any of the six ethers in the screening set allowed for excellent gallium extraction efficiencies (entries 1-6). At 6 M HCl, the ⁱPr₂O and BuOMe were found to be the best performers extracting up to 95% Ga in batch. As expected, the Ga extraction efficiency decreased substantially in 3 M HCl. Nevertheless, a 2/1 (v/v) mixture of TFT and ⁱPr₂O was able to extract 77% of Ga and only 1% of Zn (entry 3). THP co-extracted the highest amount of Zn from 6 M and 3 M HCl.

TABLE 8

| | | ZnCl₂/ 6M HCl | | ZnCl₂/ 3M HCl | |
|---|---|---|---|---|---|
| Entry | Ether | Ga extraction (%) | Zn extraction (%) | Ga extraction (%) | Zn extraction (%) |
| 1 | THP | 92 (5) | 13 (1) | 79 (1) | 15 (3) |
| 2 | Et₂O | 94 (1) | 3 (2) | 73 (16) | 4 (2) |
| 3 | ⁱPr₂O | 97 (1) | 5 (2) | 78 (1) | 1 (1) |
| 4 | Bu₂O | 89 (1) | 0.5 (1) | 20 (9) | 1 (1) |
| 5 | BuOMe | 97 (1) | 5 (2) | 48 (1) | 0.3 (1) |
| 6 | HexOMe | 93 (3) | 0.9 (1) | 26 (4) | 0.2 (1) |
| 7 | Am₂O | 79 (6) | 0.3 (1) | 11 (2) | 2 (1) |

The batch extraction of Ga and Zn for each of the dialkyl ethers in a 1:2 ratio with TFT, and hydrochloric acid, also containing 7 m zinc chloride. The figures in parentheses following the percentages are the standard deviations obtained over three runs of the extractions.

### Example 10—the Liquid Liquid Extraction in Flow of Ga and Zn Using Several Dialkyl Ethers, TFT, and Hydrochloric Acid, Also Containing 7 m Zinc Chloride, Followed by Back-Extraction of Ga into 0.1 M HCl

Next, we translated the batch experiments into fully continuous flow experiments using the apparatus depicted in FIG. 10.

The aqueous phase was formed by a 7 m ZnCl₂/3 M HCl mixture and the organic phase consisted of a 2/1, (v/v) mixture of TFT used as an interfacial tension modifier and the series of ethers were used as the extractant. The aqueous and the organic phases were combined through a tee and mixed with two static mixers and mixing tubing. The aqueous phase was retained by the membrane, while the organic phase permeated through the membrane. For the membrane-based separator, we used optimized conditions established in the previously described work on ⁴⁵Ti separation: flowrate org/aq, (mL/h)=45/15; 0.2 μm membrane pore size, 0.002″ (0.051 mm) diaphragm, and 108 cm mixing tube. The ⁶⁸Ga was selectively extracted into the organic phase. The organic phase can then be either collected or directly re-routed into the second separation module, where 0.1 M HCl was used as the aqueous phase. After the second stage LLEF, ⁶⁸Ga was selectively back-extracted into the aqueous phase together with the residual Zn.

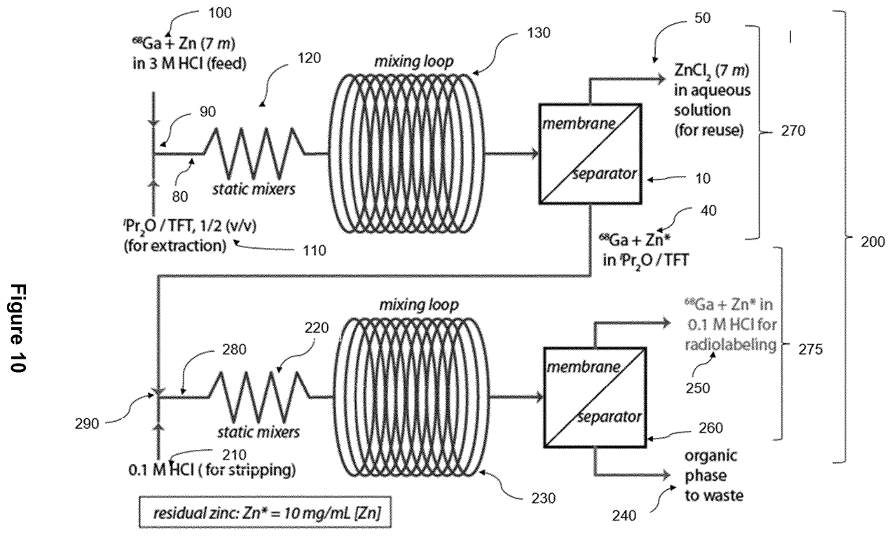

FIG. 11 shows that THP/TFT mixture was the best Ga extractant, but it also extracted the highest amount of Zn. Similar to the batch extraction, iPr₂O/TFT was the best overall performer extracting around 80% of Ga and 1.7% of Zn in flow.

Table 9 shows that gallium stripping was uniformly high (>90%) across the series. On the other hand, little selectivity was observed for Zn stripping, so that a single-stage LLEF/back-extraction sequence delivered the desired gallium solution in 0.1 M HCl containing as much as 10 mg/mL of zinc (the presence of greater than 10 mg/mL of Zn is indicated in FIGS. 10 and 12 as Zn*).

TABLE 9

| Ether | Ga stripping % | Zn stripping % |
|---|---|---|
| Et₂O | 99 | 91 |
| Pr₂O | Run 1: 93 | Run 1: 58 |
| | Run 2: 98 | Run 2: 93 |
| BuOMe | Run 1: 98 | Run 1: 47 |
| | Run 2: 98 | Run 2: 76 |
| THP | 99 | 98 |
| HexOMe | 97 | 72 |

Stripping of gallium and zinc from R₁OR₂/TFT, ½ (v/v) using 0.1M HCl.

### Example 11—the Two-Stage Liquid-Liquid Extraction in Flow of Ga and Zn Using Diisopropyl Ether, TFT, and Hydrochloric Acid, Also Containing 7 m Zinc Chloride Followed by Back-Extraction of Ga into 0.1 M HCl

To decrease the amount of co-extracted Zn, two consecutive (two-stage) liquid-liquid extractions/back-extractions in flow were performed. In this experiment, two extraction/back-extraction modules can be combined. After the first stage of extraction/back-extraction, the mixture Ga containing residual zinc was acidified to 6 M HCl and subjected to the second stage of extraction/back-extraction under analogous conditions.

After the second stage, the 71% of original gallium was recovered in the final solution for radiolabeling, which also contained 100 μg/mL of Zn.

TABLE 10
The two-stage LLEF of Ga and Zn using the mixture of dialkyl ethers,
TFT, and hydrochloric acid, also containing 7 m zinc chloride

| | 1^{st} stage extraction/ | | 2^{nd} stage extraction/ | | 1^{st} and 2^{nd} stages | |
|---|---|---|---|---|---|---|
| | back-extraction | | back-extraction | | combined | |
| | Extraction | Stripping | Extraction | Stripping | Extraction | Stripping |
| | % | % | % | % | % | % |
| Ga | 74 | 98 | 99 | 99 | 73% | 97% |
| Zn | 1 | 96 | 3 | 49 | 0.03 | 47% |

### Example 12—the Two-Stage Liquid-Liquid Extraction in Flow of Ga and Zn Using Several Dialkyl Ethers, TFT, and Hydrochloric Acid, Also Containing 7 m Zinc Chloride Also Including Scrubbing of Residual Zn with 0.1 M HCl and Back-Extraction of Ga into 0.1 M HCl

Even higher purity gallium solution can be obtained, if an extra step of liquid-liquid extraction of Zn using 8 M HCl is included (FIG. 12). This scrubbing process is selective with respect to zinc. Scrubbing removes 95% of zinc and 0.3% of gallium from the organic phase. After two LLEF/stripping and scrubbing stages, 70% of original gallium was recovered in the final solution for radiolabeling, which also contained 8 μg/mL of Zn. Scrubbing was found to be useful immediately following the first extraction step. Subsequent scrubbing steps were not found to be necessary following any subsequent extraction steps.

### Example 13—the Single-Stage Liquid-Liquid Extraction in Flow of Ga and Zn Using Diisopropyl Ether, TFT, and Hydrochloric Acid, Also Containing 7 m Zinc Chloride from a Cyclotron Target Solution

Radioqallium production: Approximately 3.5 ml of target solution (5 M ZnCl₂ in 3 M aq. HCl) was loaded in a GE PETtrace liquid target. The target chamber was made of niobium to limit corrosion. The target front foil was 250 μm niobium foil, bringing the proton energy down to 12.5 MeV from the nominal 16.5 MeV. The target was not pressurized, but left open to ensure no pressure buildup in the chamber. Bombardment was performed at a current of 5 μA for 6 minutes. After irradiation the produced Ga-68 was quantified by gamma spectroscopy on a 10% GeLi detector, calibrated using certified Eu-152 and Ba-133 sources. The produced activity at saturation was calculated to 204 MBq/μA.

Liquid-liquid extraction of radioqallium followed by irradiated target solution purification: 2.5 mL of the irradiated target solution was used as the aqueous phase for LLE. iPr₂O/TFT (1/2) was used as the organic phase. The phases were separated using the membrane separator with a 0.2 μm PTFE/PP membrane and a 2 mil (0.0508 mm) diaphragm. The aqueous flow rate was 0.25 mL/min and the organic flow rate was 0.75 mL/min. Samples of the aqueous and organic phase after the LLE were collected and the activity of ^{66,67,68}Ga (radiogallium) was measured with gamma spectroscopy. 57% of radiogallium was extracted into the organic phase. The aqueous phase was then passed through a C18 cartridge after the LLE, in order to remove trace organic solvent, and used directly for a second irradiation.

### Example 14—the Single-Stage Liquid-Liquid Extraction in Flow of Ga and Zn Using Diisopropyl Ether, TFT, and Hydrochloric Acid, Also Containing 7 m Zinc Chloride from a Re-Used Cyclotron Target Solution

Radioqallium production from a re-used cyclotron target solution: Approximately 3.5 ml of a 1:1 target solution (5 M ZnCl₂ in 3 M aq. HCl) and LLE-purified target solution from the first bombardment (Example 13) was loaded in a GE PETtrace liquid target. The target chamber was made of niobium to limit corrosion. The target front foil was 250 μm niobium foil, bringing the proton energy down to 12.5 MeV from the nominal 16.5 MeV. The target was not pressurized, but left open to ensure no pressure buildup in the chamber. Bombardment was performed at a current of 5 μA for 5 minutes. After ended irradiation the produced Ga-68 was quantified by gamma spectroscopy on a 10% GeLi detector, calibrated using certified Eu-152 and Ba-133 sources. The produced activity at saturation was calculated to 258 MBq/μA.

Liquid-liquid extraction of radiogallium from the re-used irradiated target solution: The LLE procedure described in Example 13 was used to extract Ga from 2.5 ml of the target solution from the second bombardment, which led to a radiogallium extraction of 62%.

Separation of Cu and Ni

Materials

Hydrochloric acid (37%, ACS reagent), zinc chloride (≥98%), trioctylphosphine oxide (≥98.5%), cobalt chloride, iron chloride, silver chloride, copper (II) chloride and toluene were purchased from Sigma Aldrich. High purity hydrochloric acid (37%, “Ultrapur”) was purchased from Merck. Heptane (99.7%) and hexane were purchased from VWR Chemicals. Nickel-64 (99.6% isotope-enriched) was purchased from Campro Scientific. All purchased chemicals were used without further purification.

Pall PTFE membranes were used for all experiments (47 mm diameter, 0.1/0.2/0.5 μm pore size, polypropylene support). Perfluoroalkoxy alkane (PFA) diaphragms (0.001″/0.002″/0.005″ (0.0254/0.0508/0.1270 mm)) were purchased from McMaster Carr. All PFA tubing ( 1/16″ (1.5875 mm) OD, 0.03″ (0.762 mm) ID) was purchased from Idex Health and Science. PTFE static mixers were purchased from Stamixco. The 15 mL plastic centrifuge tubes with screw caps were purchased from VWR.

Radionuclide Production and Separation

For all experiments, the cyclotron target material was Nickel-64 (99.6% isotope-enriched).

Copper-64 Production and Purification

Production: ⁶⁴Cu was produced via the ⁶⁴Ni(p,n)⁶⁴Cu reaction using a water-cooled solid target mounted on the beam line of a PETtrace (GE Healthcare) cyclotron. The target consisted of approximately 80 mg of ⁶⁴Ni metal (enriched to 99%) electroplated on a silver disk backing. The target was irradiated with a proton beam with an incident energy of 16.5 MeV and a beam current of 20 μA. After irradiation, the silver disk backing was transferred into a hot cell where it was treated with 30% HCl for 30 min. at 60° C., and then for 5 min. at 80° C., resulting in a clear green solution containing a mixture of ⁶⁴CuCl₂ and ⁶⁴NiCl₂.

Purification: The solution was decanted, diluted to 6M HCl and loaded onto a Dowex 1×8 (chloride form 200-400 mesh) column. The column was washed with 21 mL of 6 M HCl, and then with 33 mL of 5 M HCl. Finally, the column was eluted with 10 mL of 1 M HCl, which elutes ⁶⁴Cu. Final evaporation from aqueous HCl yielded 2-6 GBq of ⁶⁴Cu as ⁶⁴CuCl₂ with specific activity, 300-3000 TBq/mmol and radionuclidic purity of 99%.

Instrumentation and Methods

⁶⁴Cu was quantified by measuring radioactivities from ⁶⁴Cu radioisotopes using the CRC-55tR, CII (Capintec, Inc) dose calibrator and Princeton Gammatech LGC 5 and Ortec GMX 35195-P gamma spectrometers. Cu, Ni, Ag, Fe, Co and Zn were quantified by ICP.

An Eppendorf 5702 centrifuge was used to assist in phase separation. The membrane separator module was similar to those manufactured by Zaiput Flow Technologies. The solutions for the continuous membrane-based separation were pumped using either the KDS 100 Legacy Syringe (radioactive experiments) or the Harvard Apparatus PHD 2000 Programmable and Infusion syringe pumps (non-radioactive experiments). For batch experiments, the phase mixing was performed using an IKA ROCKER 3D digital shaker.

The continuous liquid-liquid extraction and phase separation in flow was performed using a membrane-based separator with a PFA diaphragm for integrated pressure control. A flow schematic of the apparatus is depicted in FIG. 1. The aqueous and the organic phases were combined through a tee and mixed with two static mixers and mixing tubing. The aqueous phase was retained by the membrane, while the organic phase permeated through the membrane. Under the conditions of direct extraction, the radionuclide (⁶⁴Cu) was selectively extracted over ⁶⁴Ni into the organic phase.

### Example 15—Phase Separation Performance and Liquid-Liquid Extraction in Flow of Copper and Copper-64 Using Trioctylphosphine Oxide (TOPO) in Toluene, Hexane, and Heptane, Also Containing Various Amounts of Cu, Ni, Co, Zn, Fe, and Ag in 6M Hydrochloric Acid

A single stage liquid-liquid extraction in flow of copper and copper-64 was performed using the setup depicted in FIG. 1B. Table 11 shows the results of copper and/or copper-64 extraction. Entries 1 and 2 relate to the extraction of Cu ions from a 6 M solution of HCl, also containing 0.001 M CuCl₂ and NiCl₂. Entry 3 relates to extraction of Cu ions from a 6 M solution of HCl, containing 60 ppm of Cu, Ni, Co, Zn, Fe, and Ag (mixture purchased as an ICP standard to represent typical impurities obtained during cyclotron preparation of ⁶⁴Cu from a solid ⁶⁴Ni target). Entry 4 relates to the extraction of ⁶⁴Cu ions from a 6 M solution of HCl containing a picomolar amount of ⁶⁴Cu, and no Ni or other metal ions. Trioctylphosphine oxide (TOPO) in different non-polar solvents (toluene, hexane, and heptane) was used as extractant. Using 0.2 μm PTFE membrane and 2 mil PFA diaphragm allowed for complete phase separation at various combinations of aqueous and organic flow rates.

TABLE 11
Phase separation performance and LLE in flow of copper and copper-64
from 6M HCl containing various amounts of metal impurities.

| | | | | | Flow rate | Flow rate | |
|---|---|---|---|---|---|---|---|
| | | Diaphragm | | Organic | (aqueous) | (organic) | Separation |
| Entry | Membrane | (mm) | Aqueous phase | phase | (mL/min) | (mL/min) | performance |
| 1 | 0.2 μm | 0.0508 | 6M HCl | 0.4M | 0.05 | 0.15 | Complete |
| | PTFE/PP | (2 mil) | 0.001M CuCl₂ | TOPO in | 0.10 | 0.10 | phase |
| | | | 0.01M NiCl₂ | toluene | 0.0333 | 0.1667 | separation |
| 2 | 0.2 μm | 0.0508 | 6M HCl | 0.1M | 0.05 | 0.15 | Complete |
| | PTFE/PP | | 0.001M CuCl₂ | TOPO in | 0.25 | 0.75 | phase |
| | | | 0.01M NiCl₂ | hexane | | | separation |
| 3 | 0.2 μm | 0.0508 | 60 ppm | 0.1M | 0.05 | 0.15 | Complete |
| | PTFE/PP | | Cu, Ni, Co, | TOPO in | | | phase |
| | | | Zn, Fe and | heptane | | | separation |
| | | | Ag in 6M | | | | |
| | | | HCl | | | | |
| 4 | 0.2 μm | 0.0508 | ⁶⁴Cu in 6M | 0.1M | 0.05 | 0.15 | Complete |
| | PTFE/PP | | HCl | TOPO in | 0.25 | 0.75 | phase |
| | | | | heptane | | | separation |

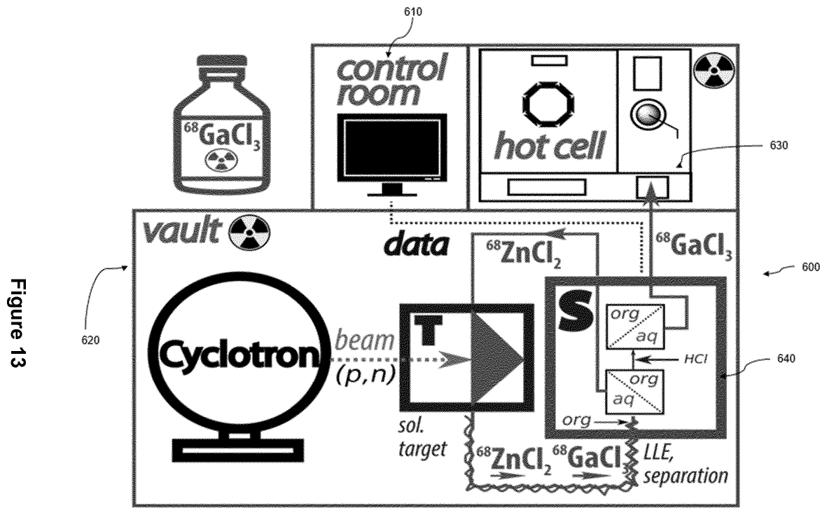

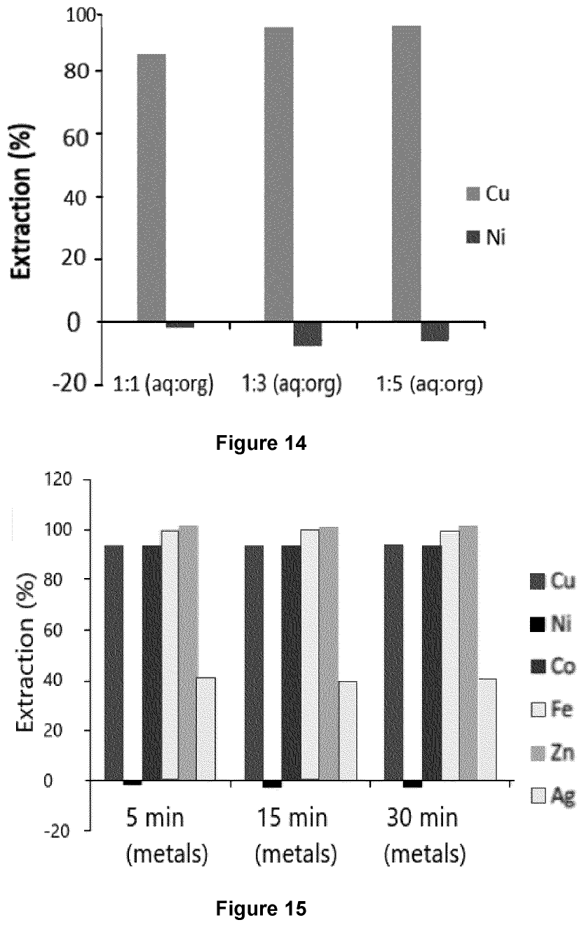

With 0.4 M TOPO in toluene up to 93% copper extraction was achieved with ratios of 1:3 and 1:5, while the extraction was 85% with a 1:1 ratio (FIG. 14). All nickel remained in the aqueous phase. The Ni data in FIG. 14 is obtained by taking the difference between the initial concentration and the concentration after extraction. As these were essentially the same, the experimental error in the measurement in some cases gives rise to a negative value shown in FIG. 14.

Extraction with 0.4 M TOPO is not sensitive to a change in interfacial tension modifier (toluene vs. hexane) and remains efficient (93%) when the concentration of TOPO is decreased to 0.1 M. No nickel is extracted to the organic phase.

FIG. 15 shows that while 0.1 M TOPO in heptane is highly selective with respect to Ni, it provides very limited selectivity with respect to Co, Fe, Zn and Ag. Thus, the ⁶⁴Cu containing solution, if obtained by proton bombardment of a solid ⁶⁴Ni target, may require further purification before use in preparing a radiopharmaceutical, if any of these metal ions would interfere with the preparation of the desired radiopharmaceutical.

Whilst the invention has been described with reference to preferred embodiments, it will be appreciated that various modifications are possible within the scope of the invention.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged herein are hereby incorporated by reference. No acknowledgement of any prior published document herein should be taken to be an admission or representation that the teaching thereof was common general knowledge in Australia or elsewhere at the date hereof.

### REFERENCES

1. Huang, R.; Wang, M.; Zhu, Y.; Conti, P.; Chen, K. Current Topics in Medicinal Chemistry 2015, 15, 795.
2. Brasse, D., Nonat, A. Dalton Trans. 2015, 44 (11), 4845.
3. Ramogida, C. F.; Orvig, C. Chemical Communications 2013, 49 (42), 4720.
4. Zeglis, B. M.; Houghton, J. L.; Evans, M. J.; Viola-Villegas, N.; Lewis, S. J. Inorg. Chem. 2014, 53 (4), 1880.
5. Martiniova, L.; Palatis, L. D.; Etchebehere, E.; Ravizzini, G. Current Radiopharmaceuticals 2016, 9 (3), 187.
6. Witowska-Patena, E.; Mazurek, A.; Dziuk, M. Central European Journal of Urology 2017, 70 (1), 37.
7. Jailian, A. R. Iranian Journal of Nuclear Medicine 2016, 24 (1), 1.
8. Eder, M.; Neels, O.; Müller, M.; Bauder-Wüst, U.; Remde, Y.; Schsfer, M.; Hennrich, U.; Eisenhut, M.; Afshar-Oromieh, A.; Haberkorn, U.; et al. Novel Preclinical and Radiopharmaceutical Aspects of [68Ga]Ga-PSMA-HBED-CC: A New PET Tracer for Imaging of Prostate Cancer. Pharmaceuticals 2014, 7 (7), 779.
9. Velikyan, I. Molecules 2015, 20, 12913-12943.
10. Chakravarty, R.; Chakraborty, S.; Radhakrishnan, E.; Kamaleshwaran, K.; Shinto, A.; Dash, A. Nuclear Medicine and Biology 2017, 46, 1.
11. Filosofov, D. V.; Garland, M.; John, K. D.; Knapp Jr., F. F.; Kunetsoz, R.; Mausner, L.; Mirzadeh, S.; Pillai, M. R. A.; Ponsard, B.; Rösch, F. IAEA Radioisotopes and Radiopharmaceuticals Series No 2, 1.
12. Chakravarty, R.; Chakraborty, S.; Ram, R.; Vatsa, R.; Bhusari, P.; Shukla, J.; Mittal, B. R.; Dash, A Journal of Labelled Compounds and Radiopharmaceuticals 2016, 59 (3), 87.
13. Engle, J. W.; Lopez-Rodriguez, V.; Gaspar-Carcamo, R. E.; Valdovinos, H. F.; Valle-Gonzalez, M.; Trejo-Ballado, F.; Severin, G. W.; Barnhart, T. E.; Nickles, R. J.; Avila-Rodriguez, M. A. Applied Radiation and Isotopes 2012, 70 (8), 1792.
14. Jensen, M.; Clark, J. In Proceedings of 13^{th} International Workshop on Targetry and Target Chemistry; Danmarks Techniske Universitet, Risø Nationallaboratioriet for Bæredygtig Energi, 2011, pp 288-292.
15. Pandey, M. K.; Byrne, J. F.; Jiang, H.; Packard, A. B.; DeGrado, T. R. American Journal of Nuclear Medicine and Molecular Imaging 2014, 4 (4), 303.
16. WO2015/175972
17. Merrill, J. C.; Lambrecht, R. M.; Wolf, A. P. International Journal of Applied Radiation and Isotopes 1978, 29, 115.
18. Sadheghi, M.; Enferadi, M.; Nadi, H. Radiochemistry 2011, 53, 411.
19. Fazaeli, Y.; Abouzadeh, M.; Aardaneh, K.; Kakavand, T.; Bayat, F.; Yousafi, K. Nuclear Technology and Radiation Protection 2014, 29, 28.
20. Severin, G. W.; Nielsen, C. H.; Jensen, A. I.; Fonslet, J.; Kjor, A.; Zhuravlev, F. J. Med. Chem 2015, 58, 7591.
21. Ishiwata, K.; Ido, T.; Monma, M.; Murakami, M.; Fukuda, H.; Kameyama, M.; Yamada, K.; Endo, S.; Yoshioka, S.; Sato. T.; Matsuzawa, T. International Journal of Radiation Applications and Instrumentation Part A: Applied Radiation and Isotopes 1991, 42, 707.
22. Vāvere, A.; Laforest, R.; Welch, M. J. Nuclear Medicine and Biology 2005, 32, 117.
23. Vāvere, A.; Welch, M. J. Journal of Nuclear Medicine 2005, 46, 683.
24. Gagnon, K.; Severin, G. W.; Barnhart, T.; Engle, J. W.; Valdovinos, H. F.; Nickles, R. J. in AIP Conference Proceedings, AIP, 2012, vol 1509, pp 211-214.
25. Severin, G. W.; Engle, J. W.; Barnhart, T. E.; Nickles, R. J. ⁸⁹Zr Radiochemistry for Positron Emission Tomography. Medicinal Chemistry 2011, 7 (5).
26. Dilworth, J. R.; Pascu, S. I. The Chemistry of PET Imaging with Zirconium-89. Chemical Society Reviews 2018, 47 (8), 2554-2571.
27. Wright, B. D.; Whittenberg, J.; Desai, A.; DiFelice, C.; Kenis, P. J. A.; Lapi, S. E.; Reichert, D. E. Microfluidic Preparation of a ⁸⁹Zr-Labeled Trastuzumab Single-Patient Dose. Journal of Nuclear Medicine 2016, 57 (5), 747-752.
28. Meijs, W. E.; Herscheid, J. D. M.; Haisma, H. J.; Wijbrandts, R.; van Langevelde, F.; Van Leuffen, P. J.; Mooy, R.; Pinedo, H. M. Production of Highly Pure No-Carrier Added ⁸⁹Zr for the Labelling of Antibodies with a Positron Emitter. Applied Radiation and Isotopes 1994, 45 (45), 1143-1147.
29. Sadeghi, M.; Kakavand, T.; Rajabifar, S.; Mokhtari, L.; Rahimi-Nezhad, A. Nukleonica 2009, 54 (1), 25.
30. Lahiri, S.; Banerjee, S.; Das, N. R. Journal of Radioanalytical and Nuclear Chemistry 1997, 218 (2), 215.
31. Nakamura, T.; Sakai, A.; Nishihama, S.; Yoshizuka, K. Solvent Extraction and Ion Exchange 2009, 27 (4), 501.
32. Mane, C. P.; Anuse, M. A. Journal of the Chinese Chemical Society (Taipei, Taiwan) 2008, 55 (4), 807.
33. S. A. Kandil, B. Scholten, Z. A. Saleh, A. M. Youssef, S. M. Qaim, H. H. Coenen, Journal of Radioanalytical and Nuclear Chemistry 2007, 274, 1, 45-52.
34. Dejesus, O. T.; Nickles, R. J. Production and Purification of ⁸⁹Zr, a Potential PET Antibody Label. International Journal of Radiation Applications and Instrumentation. Part A, Applied Radiation and Isotopes 1990, 41 (8), 789-790.
35. SATO, T. THE EXTRACTION OF ZIRCONIUM(IV) FROM AQUEOUS ACID SOLUTIONS BY TRIOCTYLPHOSPHINE OXIDE. Solvent Extraction and Ion Exchange 1983, 1 (2), 251-261.
36. X. Fan, D. J. Parker, M. D. Smith, A. Ingram, Z. Yang, J. P. K. Seville, Nuclear Medicine and Biology 2006, 33, 7, 929-944.
37. K. S. Bhatki, A. T. Rane, M. B. Kabadi, Journal of Radioanalytical Chemistry, 1969, 2, 1-2, 73-77.
38. S. Banerjee, S. Lahiri, S. B. Manohar, A. Ramaswami, Applied Radiation and Isotopes, 2002, 56, 4, 571-575.
39. M. Sadeghi and B. Shirazi, Applied Radiation and Isotopes, 2008, 66, 12, 1810-1813.
40. Y. Wen, Z. Qin, W. Liu, Lanzhou Daxue Xuebao, Ziran Kexueban 2002, 38, 1, 58-61.
41. A. Gopalakrishna, H. Naik, S. V. Suryanarayana, Y. Naik, V. T. Nimje, B. k. Nayak, S. K. Sarkar, S. Padmanabhan
42. D. Nayak and S. Lahiri Applied Radiation and Isotopes 2001, 54, 2, 189-193
43. A. Alian Nat. Bur. Stand. (US) Spec. Publ. 1969, 312, 1, 473-474
44. M. Saeki, Y. Sasaki, A. Yokoyama, JAEA-Conf 2010. 2009-007 (Ronbunshu—Nippon Genshiryoku Kenkyu Kaihatsu Kiko Ryoshi Bimu Oyo Kenkyu Bumon Dai 9-kai Hikari Ryoshi Kagaku Kenkyu Shinpojumu, 2008) 121-124.
45. K. Aardaneh, T. N. van der Walt, Journal of Radioanalytical and Nuclear Chemistry 2006, 268, 1, 25-32.
46. M. K. Das, Madhusmita, S. Chattopadhyay, S. S. Das, L. Barua, M. A. Neyar, U. Kumar, A. De Journal of Radioanalytical and Nuclear Chemistry 2016, 310, 1, 423-432.
47. J. Britton and T. F. Jamieson, Nature Protocols 2017 12, 11, 2423.
48. A. Adamo, P. L. Heider, N. Weeranoppanant, K. F. Jensen Industrial and Engineering Chemistry Research 2013, 52, 10802-10808.
49. A. G. Gaikwad and A. M. Rajput Journal of Rare Earths 2010 28, 1, 1-6.
50. R. C. Luckay, X. Sheng, C. E. Strasser, H. G. Raubenheimer, D. A. Safin, M. G. Babashkina, A. Klein Dalton Transactions 2009 39, 8227-8236.
51. J. Konczyk, A. Nowik-Zajac, C. A. Kozlowski Separation Science and Technology 2016 51, 14, 2394-2410.
52. K. Masuda, T. Ichitsuka, N. Koumura, K. Sato, S. Kobayashi, Tetrahedron, 2018, 74, 1705-1730.
53. G. Glotz, R. Lebel, D. Dallinger and C. O. Kappe, Angewandte Chemie Intl Edn 2017, 56, 13786-13789.
54. B. Leforestier, M. Vögtle Synlett 2016, 27, 1957-1962.
55. C. Battilocchio, G. Iannucci, S. Wang, E. Godineau, A. Kolleth, A. De Mesmaeker and S. V. Ley React. Chem. Eng. 2017, 2, 295-298.
56. J. Salaklang, V. Maes, M. Conradi, R. Dams, T. Junkers React. Chem. Eng. 2018, 3, 41-47.
57. F. J. Strauss, D. Cantillo, J. Guerra, C. O. Kappe React. Chem. Eng. 2016, 1, 472-476.
58. H. Yang, B. Martin and B. Schenkel Organic Process Research and Development 2018, 22, 446-456.
59. P. Zhang, N. Weeranoppanant, D. A. Thomas, K. Tahara, T. Stelzer, M. G. Russell, M. O'Mahony, A. S. Myerson, H. Lin, L. P. Kelly, K. F. Jansen, T. F. Jamieson, C. Dai, Y. Cui, N. Briggs, R. L. Beingessner and A. Adamo, Chem. Eur. J. 2018, 24, 2776-2784.
60. Y. Shen, N. Weeranoppanant, L. Xie, Y. Chen, M. R. Lusardi, J. Imbrogno, M. G. Bawendi and K. F. Jansen, Nanoscale 2017, 9, 7703-7707.
61. H. P. L. Gemoets, G. Laudadio, K. Verstraete, V. Hessel and T. Nosl, Angewandte Chemie Intl Edn 2017, 56, 7161-7165.
62. E. Y. Tshuva, I. Goldberg and M. Kol, J. Am. Chem. Soc. 2000, 122, 10706-10707.
63. T. A. Immel, M. Grutzke, A.-K. Spate, U. Groth, P. Ohlschlager and T. Huhn, Chem. Commun. 2012, 48, 5790-5792.
64. J. Siikanen, The Journal of Nuclear Medicine, 54, 1095.
65. R. Mondal and P. Tarafder, Microchimica Acta, Mikrochimica Acta, Microchim. Acta, Microch Act, Microchim Acta, Mikrochim Acta, Mikroch Act, Mikrochimica Acta Micro and Trace Analysis, Microchimica Acta, 2004, 148, 327-333.
66. E. de Wolf, G. van Koten and B.-J. Deelman, Chemical Society Reviews, 1999, 28, 37-41.
67. P. Sobota, K. Przybylak, J. Utko, L. Jerzykiewicz, A. Pombeiro, M. da Silva and K. Szczegot, Chemistry—a European Journal, 2001, 7, 951-958.
68. Swift, E. H. A NEW METHOD FOR THE SEPARATION OF GALLIUM FROM OTHER ELEMENTS. J. Am. Chem. Soc. 1924, 46 (11), 2375-2381.
69. Irving, H. M.; Rossotti, F. J. C. The Solvent Extraction of Group IIIB Metal Halides. Analyst 1952, 77 (920), 801-812.
70. Nachtrieb, N. H.; Fryxell, R. E. The Extraction of Gallium Chloride by Isopropyl Ether. J. Am. Chem. Soc. 1949, 71 (12), 4035-4039.
71. BROOKS, R. R.; LLOYD, P. J. Influence of Molecular Structure on the Liquid/Liquid Extraction of the Chloro-Complexes of Gallium and Indium with Aliphatic Ethers. Nature 1961, 189, 375.
72. Kirchnerová, J.; Cave, G. C. B. The Solubility of Water in Low-Dielectric Solvents. Canadian Journal of Chemistry 1976, 54 (24), 3909-3916.
73. EP3101660.
74. Tolmachev, V; Lundqvist, H; Einarsson, L. Production of ⁶¹Cu from a natural nickel target Appl. Radiat. Isot. 1998, 49(1-2), 79-81.
75. Vāvere, A. L.; Lewis, J. S. Dalton Transactions, 2007, 4893-4902.
76. Devi, N. B.; Nayak, B. The Journal of the Southern African Institute of Mining and Technology, 2014, 114, 937-943.
77. Devi, N. B.; Mishra, S. The Journal of the Southern African Institute of Mining and Technology, 2012, 112, 859-864.
78. Sepúlveda, R.; Castille, J.; Plaza, A.; Senchez, J.; Torres, A.; Romero, J. The Journal of Supercritical Fluids 2017, 128, 26-31.
79. Djebabra, S.; Barkat, D. J Mater. Environ. Sci. 2015, 6 (11), 3382-3387.

1. A method of separation of a radiometal ion from a target metal ion, comprising a first liquid-liquid extraction step in which an organic phase comprising an extractant and an interfacial tension modifier is mixed with an aqueous phase comprising the radiometal ion and the target metal ion in order that the radiometal ion is at least partially transferred to the organic phase, followed by a first phase separation step, wherein the phase separation is carried out in flow comprising the use of a microfiltration membrane to separate the phases based on the interfacial tension between the phases such that a permeate phase passes through the membrane and a retentate phase does not, wherein:
a. the radiometal ion is a ⁶⁸Ga ion, the target metal ion is a ⁶⁸Zn ion, the extractant is selected from one or more dialkyl ethers R¹OR², wherein the two alkyl groups R¹ and R² can be the same or different, or can together form a cyclic ether, and can optionally be substituted, and the interfacial tension modifier is selected from one or more aromatic hydrocarbons, which may optionally be halogenated, and/or one or more C2-C9 alkanes, which may optionally be halogenated; or
b. the radiometal ion is a ⁸⁹Zr ion, the target metal ion is a ^{nat}Y ion, the extractant is a solvent able to function as a bidentate ligand for ⁸⁹Zr via two oxygen atoms, and the interfacial tension modifier is a solvent having similar properties to the extractant, but that are not able to function as a bidentate ligand for the ⁸⁹Zr ion, such that it does not interfere with the ability of the extractant to interact with the ⁸⁹Zr ions; or
c. the radiometal ion is a ⁴⁵Ti ion, the target metal ion is a ^{nat}Sc ion, the extractant is a solvent able to function as a bidentate ligand for ⁴⁵Ti via two oxygen atoms, and the interfacial tension modifier is a solvent having similar properties to the extractant, but that are not able to function as a bidentate ligand for the ⁴⁵Ti ion, such that it does not interfere with the ability of the extractant to interact with the ⁴⁵Ti ions; or
d. the radiometal ion is a ⁶⁴Cu ion, the target metal ion is a ⁶⁴Ni ion, the extractant is selected from: one or more trialkyl phosphine oxides; one or more alkylphosphoric acid monoalkyl esters; one or more diketones having the structure R³—C(═O)CH₂C(═O)—R⁴, in which R³ and R⁴ are each independently an alkyl or an aryl group; and one or more aldoximes or ketoximes in which the substituent(s) of the oxime group are aromatic groups; and the interfacial tension modifier is a solvent comprising one or more straight or branched chain cyclic or acyclic aliphatic alkanes having from five to sixteen carbon atoms, which may optionally be substituted, and/or a solvent comprising one or more aromatic hydrocarbons, which may optionally be substituted.

2. (canceled)

3. (canceled)

4. The method according to claim 1, wherein the first liquid-liquid extraction step is conducted in flow, and wherein the first liquid-liquid extraction step comprises mixing the aqueous phase and the organic phase such that stable liquid-liquid segmented flow of the mixture is established.

5. (canceled)

6. The method according to claim 1, wherein the aqueous phase comprises a concentration of aqueous hydrochloric acid or nitric acid of greater than or equal to 3M.

7. (canceled)

8. (canceled)

9. The method according to claim 1, wherein the radiometal ion and the target metal ion are defined as follows:
a. the radiometal ion is a ⁶⁸Ga(III) ion and the target metal ion is a ⁶⁸Zn(II) ion; or
b. the radiometal ion is a ⁸⁹Zr(IV) ion and the target metal ion is a ^{nat}Y(III) ion; or
c. the radiometal ion is a ⁴⁵Ti(IV) ion and the target metal ion is a ^{nat}Sc(III) ion; or
d. the radiometal ion is a ⁶⁴Cu(II) ion and the target metal ion is a ⁶⁴Ni(TI) ion.

10. The method according to claim 1, wherein the radiometal ion is a Ti ion and the target metal ion is a Sc ion, and: the aqueous phase is a solution in 12M HCl; the extractant is selected from the group consisting of maltol, vanillin, eugenol, and guaiacol (o-methoxyphenol); and the interfacial tension modifiers are selected from the group consisting of fluorobenzene, trifluorotoluene, thiophene and anisole.

11. The method according to claim 10, wherein the extractant is guaiacol and the interfacial tension modifier is anisole.

12. (canceled)

13. (canceled)

14. The method according to claim 1, wherein the radiometal ion is a Ga ion and the target metal ion is a Zn ion, the extractant is selected from the group consisting of diethylether, butylmethyl ether, diisopropyl ether, tetrahydropyran, methyl hexyl ether, dibutyl ether and diamyl ether, and the interfacial tension modifier is selected from the group consisting of: a fluorinated aromatic hydrocarbon; an aromatic hydrocarbon; an alkoxybenzene; a halogenated alkane; and an alkane.

15. The method according to claim 14, wherein the aqueous phase is a solution in 6M HCl, and the extractant is selected from diethyl ether, diisopropyl ether, dibutyl ether, butyl methyl ether and hexyl methyl ether.

16-18. (canceled)

19. The method according to claim 14, wherein the extractant is selected from butyl methyl ether, diisopropyl ether, dibutyl ether and diethyl ether, and the interfacial tension modifier is selected from the group consisting of toluene, anisole, 1,2-dichloroethane, trifluorotoluene and heptane.

20-22. (canceled)

23. The method according to claim 14, further comprising a back extraction procedure comprising, following the first phase separation step, a first back-extraction step in which an organic phase comprising the radiometal ion is mixed with an aqueous solution of a protic acid in order that the radiometal ion is at least partially transferred to the aqueous solution, followed by a back-extraction phase separation step, in which the phase separation is carried out in flow comprising the use of a microfiltration membrane to separate the phases based on the interfacial tension between the phases such that a permeate phase passes through the membrane and a retentate phase does not, in order to obtain an aqueous solution comprising the radiometal ion.

24. The method according to claim 23, wherein the aqueous solution of a protic acid is an aqueous solution of less than 6 M HCl.

25-31. (canceled)

32. The method according to claim 1, wherein the radiometal ion is a Zr ion and the target metal ion is a Y ion, the extractant is selected from the group consisting of maltol, vanillin, eugenol, and guaiacol (0-methoxyphenol), and the interfacial tension modifier is selected from the group consisting of fluorobenzene, trifluorotoluene, thiophene and anisole.

33. The method according to claim 32, wherein the extractant is guaiacol (o-methoxyphenol) and the interfacial tension modifier is anisole.

34-37. (canceled)

38. The method according to claim 1, wherein the radiometal ion is a Zr ion and the target metal ion is a Y ion, the extractant is 0.1 M trioctylphosphine oxide (TOPO), the interfacial tension modifier is hexane, and the aqueous phase is a solution in 6 M HCl.

39. The method according to claim 1, wherein the radiometal ion is a Cu ion and the target metal ion is a Ni ion, the extractant is selected from: one or more trialkylphosphine oxides in which the alkyl groups are selected from: straight chain or branched hydrocarbons having from six to ten carbon atoms; one or more alkylphosphoric acid monoalkyl esters having the structure R⁵—P(═O)(OH)—OR⁶, where R⁵ and R⁶ are each independently a branched or unbranched C₆ to C₁₀ alkyl group; one or more diketones having the structure R³—C(═O)CH₂C(═O)—R⁴, in which R³ and R⁴ are each independently an optionally halogenated branched or unbranched C₁ to C₁₀ alkyl group or a substituted or unsubstituted phenyl group; one or more aldoximes or ketoximes having an aromatic substituent wherein the benzene ring is substituted with both an oxygen and an alkyl group.

40. The method according to claim 39, in which the extractant is selected from the group consisting of: Cyanex 923 (TRPO), trioctylphosphine oxide, 2-ethylhexylphosphoric acid mono-2-ethylhexyl ester (PC-88A), 1-phenyldecane-1,3-dione, heptadecane-8,10-dione, 1,3-diphenylpropane-1,3-dione, 5-nonylsalicylaldoxime, 5-dodecylsalicylaldoxime, Acorga® P50, or 2-hydroxy-5-nonylacetophenone oxime.

41. (canceled)

42. The method according to claim 39, in which the interfacial tension modifier is selected from n-pentane, n-hexane, n-heptane, n-octane, n-nonane, n-decane, n-undecane, i-hexane, neo-hexane, i-heptane, neo-heptane, cyclohexane, cycloheptane, cyclooctane, kerosene, light petroleum, benzene, naphthalene, toluene, ethylbenzene, dimethylbenzene, iso-octane and mixtures thereof.

43-52. (canceled)

53. The method according to claim 1, wherein the microfiltration membrane is a PTFE membrane having a pore size of 0.2 Om, and the PFA diaphragm has a thickness of 0.002″ (0.0508 mm).

54. (canceled)

55. A method of generation of radiometal ions from a target metal, comprising:
a. providing a solid target metal or an aqueous solution of ions of the target metal;
b. irradiation of the solid target metal or the target metal ion solution with a particle beam to produce a solid mixture of radiometal and target metal, or a mixture of radiometal ions and target metal ions in aqueous solution
c. separation of the radiometal ions from the target metal ions according to the method of claim 1.

56-59. (canceled)

60. The method of generation of radiometal ions from a target metal according to claim 55, wherein step a. comprises providing a solid target metal, and, after step b. and before step c., the method further comprises a step of dissolution of the solid mixture of radiometal and target metal to produce an aqueous solution comprising radiometal ions and target metal ions.

61-63. (canceled)

64. A method to make a radiolabelled pharmaceutical, comprising the steps of:
a. using the method of claim 55 to provide separated radiometal ions; and
b. reacting the separated metal ions with a reactive precursor of the radiolabelled pharmaceutical in a manner to obtain the radiolabelled pharmaceutical.

65. The method of claim 64, wherein the radiometal ions comprise ⁶⁸Ga, and wherein step b. comprises using a back extraction procedure to obtain an aqueous solution comprising the separated radiometal ions, wherein the aqueous solution comprising the separated radiometal ions is reacted with the precursor of the radiolabelled pharmaceutical in a manner in step b. to obtain the radiolabelled pharmaceutical.

66. (canceled)

67. Apparatus for conducting separation of a radiometal ion from a target metal ion by means of a liquid-liquid extraction and phase separation carried out in continuous flow, comprising:
a first inlet for an aqueous phase comprising the radiometal ion and the target metal ion;
a second inlet for an organic phase comprising an extractant and an interfacial tension modifier;
one or more mixers for mixing the organic phase and the aqueous phase;
tubing to convey the mixture of the organic phase and the aqueous phase;
a phase separation apparatus comprising a microfiltration membrane to separate the organic phase from the aqueous phase based on the interfacial tension between the phases such that a permeate phase passes through the membrane and a retentate phase does not;
a first outlet for the aqueous phase exiting the phase separation apparatus;
a second outlet for the organic phase exiting the phase separation apparatus.

68-94. (canceled)

95. The method of claim 64, wherein the radiometal ions comprise ⁴⁵Ti and/or ⁸⁹Zr.
